(19) United States
(12) Patent Application Publication
(10) Pub. No.: US 2019/0090757 A1
(43) Pub. Date: Mar. 28, 2019

(54) Method and Apparatus for Estimating a Cardiovascular Characteristic Parameter, and Storage Medium for the Same

(71) Applicant: Guangdong Transtek Medical Electronics Co. Ltd., Zhongshan (CN)

(72) Inventors: Chaoliang XIAO, Zhongshan (CN); Xiqian LIN, Zhongshan (CN); Guanqun ZHANG, Zhongshan (CN)

(21) Appl. No.: 16/041,555

(22) Filed: Jul. 20, 2018

(30) Foreign Application Priority Data
Sep. 22, 2017 (CN) 2017108701450

(51) Int. Cl. A61B 5/0205; A61B 5/00; A61B 5/024

(52) U.S. Cl. CPC A61B 5/0205; A61B 5/7264; A61B 5/02405; A61B 5/0402; A61B 5/681; A61B 5/7278; A61B 5/02416

(57) ABSTRACT

A method for estimating a cardiovascular characteristic parameter comprises steps of: obtaining a first action amount of a subject and a sensing signal of a cardiovascular aspect within a first time interval, wherein the first action amount is located within a first action amount threshold range; determining, based on the first action amount threshold range, a first estimation scope for estimating the cardiovascular characteristic parameter; and determining an estimation value of the cardiovascular characteristic parameter based on the above steps.

The present disclosure estimates a cardiovascular characteristic parameter mainly based on a sensing signal, an action amount, an action amount threshold range, and a corresponding estimation scope. The action amount and the corresponding estimation scope reduces a certain computation capacity of corresponding exemplary apparatus, such as PPG sensor or ECG sensor, smart wristband, or smart watch.

### CROSS-REFERENCE TO RELATED APPLICATION

This application claims the priority to Chinese Patent Application No. 2017108701450, filed Sep. 22, 2017 in the State Intellectual Property Office of P.R. China, which is expressly incorporated by reference herein in its entirety.

### FIELD

The present disclosure relates to the field of healthcare, and more particularly to a method and an apparatus for estimating a cardiovascular characteristic parameter, and a storage medium for the same.

### BACKGROUND

Wearable devices, motion detection devices, and physiological detection devices (such as a heart rate strap, a sports band, etc.) may measure a heart rate, a blood pressure, and the like using an ECG or PPG (photoplethysmography) approach and present them to users. When a human body is less active or inactive, the measured heart rate will be relatively accurate. However, when a user has a large action amount or a wearing body part has large-amplitude or high-frequency motions, numerical values measured by the prior art are always inaccurate.

The existing solutions are mainly based on qualities of relevant signals; when a quality of a signal is poor, a measurement is compensated according to a compensation scheme. However, in practice, a relatively large individual difference is still caused: measurement values of some users would be relatively accurate, while measurement values of some other users still need to be improved. In addition, prior arts always simply compensate for a heart rate measurement or a heart rate estimation, which lack compensations for a variety of cardiovascular characteristic parameters, and the estimations thereby are less accurate.

Therefore, a heretofore unaddressed need exists in the art to address the aforementioned deficiencies and inadequacies.

### SUMMARY

To solve the technical problems above, the present disclosure provides a method for estimating a cardiovascular characteristic parameter, the method comprising steps of:

S100: obtaining a first action amount of a subject and a sensing signal of a cardiovascular aspect within a first time interval, wherein the first action amount is located within a first action amount threshold range, and the sensing signal of the cardiovascular aspect includes any one or a combination of: a photoplethysmographic signal, an electrocardiographic signal;

S200: determining, based on the first action amount threshold range, a first estimation scope for estimating the cardiovascular characteristic parameter; and

S300: determining an estimation value of the cardiovascular characteristic parameter based on the above steps.

Further, the present disclosure further provides an apparatus for estimating a cardiovascular characteristic parameter, the apparatus comprising:

a first obtaining unit configured to obtain a first action amount of a subject and a sensing signal of a cardiovascular aspect within a first time interval, wherein the first action amount is located within a first action amount threshold range, and the sensing signal of the cardiovascular aspect includes any one or a combination of: a photoplethysmographic signal, an electrocardiographic signal;

a second determining unit configured to determine, based on the first action amount threshold range, a first estimation scope for estimating the cardiovascular characteristic parameter; and

a third determining unit configured to determine an estimation value of the cardiovascular characteristic parameter based on the above unit.

In addition, the present disclosure further provides a computer-readable storage medium, wherein:

the computer-readable storage medium comprises one or more programs for executing any method mentioned above.

Furthermore, the present disclosure discloses a data processing apparatus, the data processing apparatus comprising:

the computer-readable storage medium mentioned above; and

one or more processors for executing programs in the computer-readable storage medium.

Through the technical solutions above, the present disclosure estimates a cardiovascular characteristic parameter mainly based on a sensing signal, an action amount, and a corresponding estimation scope, wherein the action amount and the corresponding estimation scope may reduce a certain computation capacity, thereby enhancing energy utilization, lowering unnecessary energy waste, and even capable of achieving a more accurate and personalized estimation of the cardiovascular characteristic parameter in a specific implementation.

### BRIEF DESCRIPTION OF THE DRAWINGS

FIG. 1 is a flow chart for a method in one embodiment of the present disclosure;

FIG. 2 is an original PPG waveform in a static state in an embodiment of the present disclosure;

FIG. 3 is an original PPG signal waveform corresponding to a first action amount in another embodiment of the present disclosure;

FIG. 4 is a heart rate curve diagram corresponding to the first action amount in a further embodiment of the present disclosure;

FIG. 5 is a heart rate curve diagram corresponding to the first action amount in a still further embodiment of the present disclosure;

Curve 1 in FIG. 4 represents heart rate search values obtained with reference to an existing heart rate search technology, which are used as reference heart rate values; Curve 3 in FIGS. 4 and 5 represents actual heart rate values; Curve 2 in FIG. 5 represents heart rate prediction values based on a fitting equation of the action amount to heart rates; and some reference heart rate values of Curve 1 in FIG. 5 have been calibrated based on the fitting equation of the action amount to the heart rates;

FIG. 6 is a first spectrogram (a spectrogram of the motion signal at a first action amount) according to another embodiment of the present disclosure;

FIG. 7 is a second spectrogram (a spectrogram of the original PPG signal at the first action amount) according to another embodiment of the present disclosure;

FIG. 8 is a third spectrogram (a spectrogram of the filtered PPG signal at the first action amount) according to another embodiment of the present disclosure; and

FIG. 9 is a schematic diagram of an apparatus according to an embodiment of the present disclosure.

### DETAILED DESCRIPTION

The present disclosure will now be described more fully hereinafter with reference to the accompanying drawings, in which exemplary embodiments of the present disclosure are shown. The present disclosure may, however, be embodied in many different forms and should not be construed as limited to the embodiments set forth herein. Rather, these embodiments are provided so that this disclosure is thorough and complete, and will fully convey the scope of the disclosure to those skilled in the art. Like reference numerals refer to like elements throughout.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. For convenience, certain terms may be highlighted, for example using italics and/or quotation marks. The use of highlighting and/or capital letters has no influence on the scope and meaning of a term; the scope and meaning of a term are the same, in the same context, whether or not it is highlighted and/or in capital letters. It is appreciated that the same thing can be said in more than one way. Consequently, alternative language and synonyms may be used for any one or more of the terms discussed herein, nor is any special significance to be placed upon whether or not a term is elaborated or discussed herein. Synonyms for certain terms are provided. A recital of one or more synonyms does not exclude the use of other synonyms. The use of examples anywhere in this specification, including examples of any terms discussed herein, is illustrative only and in no way limits the scope and meaning of the disclosure or of any exemplified term. Likewise, the disclosure is not limited to various embodiments given in this specification.

It is understood that when an element is referred to as being “on” another element, it can be directly on the other element or intervening elements may be present therebetween. In contrast, when an element is referred to as being “directly on” another element, there are no intervening elements present. As used herein, the term “and/or” includes any and all combinations of one or more of the associated listed items.

It is understood that, although the terms first, second, third, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below can be termed a second element, component, region, layer or section without departing from the teachings of the present disclosure.

It is understood that when an element is referred to as being “on,” “attached” to, “connected” to, “coupled” with, “contacting,” etc., another element, it can be directly on, attached to, connected to, coupled with or contacting the other element or intervening elements may also be present. In contrast, when an element is referred to as being, for example, “directly on,” “directly attached” to, “directly connected” to, “directly coupled” with or “directly contacting” another element, there are no intervening elements present. It are also appreciated by those of skill in the art that references to a structure or feature that is disposed “adjacent” to another feature may have portions that overlap or underlie the adjacent feature.

The terminology used herein is for the purpose of describing particular embodiments only and is not intended to be limiting of the disclosure. As used herein, the singular forms “a,” “an,” and “the” are intended to include the plural forms as well, unless the context clearly indicates otherwise. It is further understood that the terms “comprises” and/or “comprising,” or “includes” and/or “including” or “has” and/or “having” when used in this specification specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top,” may be used herein to describe one element's relationship to another element as illustrated in the figures. It is understood that relative terms are intended to encompass different orientations of the device in addition to the orientation shown in the figures. For example, if the device in one of the figures is turned over, elements described as being on the “lower” side of other elements would then be oriented on the “upper” sides of the other elements. The exemplary term “lower” can, therefore, encompass both an orientation of lower and upper, depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

Unless otherwise defined, all terms (including technical and scientific terms) used herein have the same meaning as commonly understood by one of ordinary skill in the art to which the present disclosure belongs. It is further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

As used herein, “around,” “about,” “substantially” or “approximately” shall generally mean within 20 percent, preferably within 10 percent, and more preferably within 5 percent of a given value or range. Numerical quantities given herein are approximate, meaning that the terms “around,” “about,” “substantially” or “approximately” can be inferred if not expressly stated.

As used herein, the terms “comprise” or “comprising,” “include” or “including,” “carry” or “carrying,” “has/have” or “having,” “contain” or “containing,” “involve” or “involving” and the like are to be understood to be open-ended, i.e., to mean including but not limited to.

As used herein, the phrase “at least one of A, B, and C” should be construed to mean a logical (A or B or C), using a non-exclusive logical OR. It should be understood that one or more steps within a method may be executed in different order (or concurrently) without altering the principles of the disclosure.

Embodiments of the disclosure are illustrated in detail hereinafter with reference to accompanying drawings. It should be understood that specific embodiments described herein are merely intended to explain the disclosure, but not intended to limit the disclosure. In accordance with the purposes of this disclosure, as embodied and broadly described herein, this disclosure, in certain aspects, relates to methods and systems for identification of bacteria in a biological fluid using Raman spectroscopy and applications of the same.

To provide a more comprehensive illustration of the embodiments of the present disclosure, a plurality of details will be expounded infra. However, to those skilled in the art, it is apparent that the embodiments of the present disclosure may be implemented without these details. In addition, the features in different embodiments described infra may be combined with one another, unless otherwise specifically indicated.

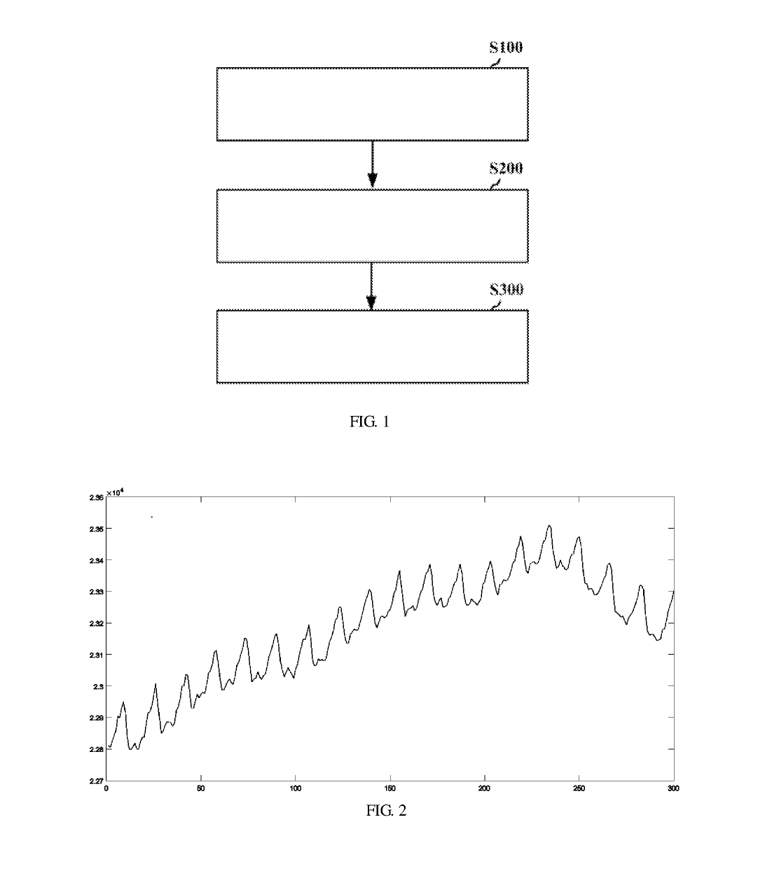

Because photoplethysmographic signals (PPG signals) or electrocardiographic signals (ECG signals) are affected during a motion process of a subject, in order to obtain a cardiovascular characteristic parameter as accurate as possible, referring to FIG. 1, one embodiment discloses a method for estimating a cardiovascular characteristic parameter, the method comprising steps of:

S100: obtaining a first action amount of a subject and a sensing signal of a cardiovascular aspect within a first time interval, wherein the first action amount is located within a first action amount threshold range, and the sensing signal of the cardiovascular aspect includes any one or a combination of: a photoplethysmographic signal, an electrocardiographic signal;

S200: determining, based on the first action amount threshold range, a first estimation scope for estimating the cardiovascular characteristic parameter; and

S300: determining an estimation value of the cardiovascular characteristic parameter based on the above steps.

The embodiment is mainly characterized in that:

(1) the embodiment determines the estimation scope according to the action amount threshold range where the action amount is located; it may be appreciated that different action amount threshold ranges theoretically may correspond to respective estimation scopes. If all data within an estimation scope is represented as a data table, each estimation scope corresponds to a lookup table area. Apparently, the correspondence between different action amount threshold ranges and their respective estimation scopes facilitates quickly determining the cardiovascular characteristic parameter based on an estimation scope, rather than determining an estimation value of the cardiovascular characteristic parameter by searching or other computation approach within a same large scope at any action amount. Apparently, presence of the estimation scope may reduce a certain computation capacity, thereby enhancing energy efficiency of the corresponding processor or sensor (for example, PPG sensor or ECG sensor) and lowering unnecessary energy waste, which is very beneficial to various types of devices, especially to small-sized devices, such as a smart wristband or smart watch with the function of a sphygmomanometer or a heart rate meter.

Exemplarily, the present disclosure may assess an action amount of the subject based on acceleration changes, which will be described in detail infra. It is easily understood that the present disclosure may also assess, based on velocity changes, the action amount of the subject and determine the corresponding estimation scope.

(2) The embodiment takes into account the impacts on the cardiovascular characteristic parameter from the action amount and the corresponding estimation scope; this means when the subject is in a motion state, the embodiment also incorporates another factor (namely, the action amount) closely associated with the cardiovascular characteristic parameter, in addition to the sensing signal of a cardiovascular aspect.

In one aspect, this embodiment adopts an innovative approach: introducing the estimation scope in the estimation of the cardiovascular characteristic parameter. As mentioned in (1), the estimation scope may enhance energy efficiency.

In the other aspect, this embodiment comprehensively considers the sensing signal of the cardiovascular aspect, the first action amount or the first motion threshold range, and the first estimation scope, such that those factors closely associated with the cardiovascular characteristic parameter are incorporated as comprehensively as possible. In other words, this embodiment facilitates enhancing the accuracy of the estimation value of the cardiovascular characteristic parameter.

As far as this embodiment is concerned, the sensing signal of a cardiovascular aspect and the first action amount may be directly obtained by a single-function sensor or a multi-sensor integrated element, or the sensing signal of the cardiovascular aspect and the first action amount may be indirectly obtained by a single-function sensor or a multi-sensor integrated element in further conjunction with a data processor. In addition, because the first estimation scope is determined based on the first action amount threshold range, it may be deemed that the first estimation scope is indirectly obtained; however, it does not mean that the first estimation scope cannot be directly obtained in the future, e.g., when a processor that may determine the first estimation scope is implemented.

In another embodiment, the cardiovascular characteristic parameter includes, but not limited to: a heart rate, a respiratory rate, an oxyhemoglobin saturation, a heart rate variability (HRV), a systolic blood pressure (also “largest pressure”), a diastolic blood pressure (also “minimum pressure”), and a blood pressure instant variability (BPIV). In the prior art, besides estimating the heart rate using the PPG signal or the ECG signal, those skilled in the art may also estimate the oxyhemoglobin saturation, the respiratory rate, the systolic pressure, and the diastolic pressure using the PPG signal. In addition, the prior art also reveals that the PPG signal or the ECG signal is not only associated with the heart rate, but also associated with the respiratory rate, the oxyhemoglobin saturation, the HRV and the BPIV.

It may be understood that analogous to the oxyhemoglobin saturation, all other cardiovascular characteristic parameters estimated using the cardiovascular PPG signal, e.g., concentration of a certain substance in blood, should also belong to the cardiovascular characteristic parameters in the present disclosure.

In a further embodiment, the first action amount includes any one of the following parameters or a combination thereof: an acceleration parameter, a velocity parameter, a pace count, and a pace frequency parameter. It is easily understood that these parameters are all associated with motions of the subject. Additionally, the subject may be a human being or other animal with a blood circulation system. it would be understood that the acceleration parameter, the velocity parameter, the pace count, and the pace frequency parameter may be obtained directly or indirectly by one or more of the following sensors: an accelerator, a gyroscope, and a GPS.

With the acceleration parameter as an example, when analyzing the acceleration data, acceleration changes may be used as one of the grounds for assessing a human body activity. When a sampling rate of a sensor that may sense the acceleration is 25 Hz, i.e., acquiring 25 pieces of data per second, by summing absolute values of differences between every two adjacent pieces of data within each second, an accumulated value a of the differences between sample values of accelerations within one second is obtained; and then an average value A of values a within every 10 seconds is resolved, where the scope of the sample values of each piece of sample data is [0, 255], and the corresponding acceleration scope is [0, 2 g], wherein g denotes a gravitational acceleration. The average value A may be used as a first action amount within the first time interval.

In the present disclosure, the first action amount threshold range may be defined as a range≤40, i.e., (0, 40], or defined as (40, 60], or (60, 100], or (100, 120], all of which belong to different action amount threshold ranges, corresponding to different action amount levels. Exemplarily, for the above different action amount threshold ranges:

(0, 40] is referred to as an almost static action amount range, corresponding to an estimation scope of 35˜85 times per minute;

(40, 60] is referred to as a relatively lower level action amount range, corresponding to an estimation scope of 45˜100 times per minute;

(60, 100] is referred to as a low level action amount range, corresponding to an estimation scope of 50˜100 times per minute; and

(100, 120] is referred to as a medium level action amount range, corresponding to an estimation scope of 70˜120 times per minute.

In other words, the estimation scopes of the present disclosure may be limited by upper and lower limit parameters. In view that the heart rates of athletes are different from those of common people, it needs to be indicated that ideally, the upper or lower limits of the estimation scopes may be updated or adjusted.

In another embodiment, the step 300 may specifically comprises the following sub-steps:

S3011: when a reliability degree of the sensing signal of the cardiovascular aspect does not meet a first threshold requirement, determining a cardiovascular characteristic reference value corresponding to the first action amount based on the first estimation scope;

S3012: using the cardiovascular characteristic reference value corresponding to the first action amount as an estimation value of the cardiovascular characteristic parameter.

As far as this embodiment is concerned, it additionally introduces the reliability degree of the sensing signal of a cardiovascular aspect over the previous embodiment. It would be appreciated that it is possible that the action amount causes a relatively large fluctuation of the sensing signal of the cardiovascular aspect, causing that the reliability degree of the sensing signal of the cardiovascular aspect does not meet the first threshold requirement, deviating from the objective condition of the cardiovascular of the subject. Likewise, it is also possible that the action amount does not cause a relatively large fluctuation of the sensing signal of the cardiovascular aspect, such that the sensing signal of the cardiovascular aspect still meets the reliability degree requirement, not deviating from the objective condition of the cardiovascular of the subject.

First, for example, when the reliability degree passes a confidence review: it would be understood that it is possible that the action amount causes a relatively large fluctuation of the sensing signal of a cardiovascular aspect, such that the sensing signal of the cardiovascular aspect becomes not meeting a confidence requirement in a statistical sense, deviating from the objective condition of the cardiovascular of the subject. Likewise, it is also possible that the action amount does not cause a relatively large fluctuation of the sensing signal of a cardiovascular aspect, such that the sensing signal of the cardiovascular aspect still meets the confidence requirement in a statistical sense, not deviating from the objective condition of the cardiovascular of the subject.

Suppose a specific confidence threshold requirement of the confidence is 0.95 or 0.99. According to the confidence theory, if the confidence does not meet the confidence threshold requirement, it belongs to a low confidence situation;

If the confidence is relatively low, it will be unnecessary to estimate the cardiovascular characteristic parameter using the sensing signal of the cardiovascular aspect. It may be understood that execution of steps S3011 and S3012 means that the embodiment does not perform any narrowly-defined estimation computation; instead, the embodiment directly determines, according to the first action amount, the cardiovascular characteristic reference value corresponding to the first action amount based on the first estimation scope, and directly uses the cardiovascular characteristic reference value as the estimation value of the cardiovascular characteristic parameter under the current action amount.

Secondly, for another example, when the reliability degree passes a signal-to-noise ratio (SNR) review: it may be understood that it is possible that the action amount causes a relatively large fluctuation of the sensing signal of a cardiovascular aspect, such that the sensing signal of the cardiovascular aspect becomes not meeting an SNR threshold requirement of the SNR, deviating from the objective condition of the cardiovascular of the subject. Likewise, it is also possible that the action amount does not cause a relatively large fluctuation of the sensing signal of the cardiovascular aspect, such that the sensing signal of the cardiovascular aspect still meets the specific SNR threshold requirement, not deviating from the objective condition of the cardiovascular of the subject.

The SNR unit is db, which is generally not directly measured, but translated from a measured noise signal amplitude. A general practice comprises: assigning a standard signal (e.g., 0.775 Vrms or 2Vp-p @ 1 kHz) to an amplifier, tuning a magnification of the amplifier such that it reaches the largest non-distortion output power or amplitude (the scope of distortion is decided by the manufacturer, usually 10%, or 1%); recording the current output amplitude Vs of the amplifier; then removing the input signal, and measuring the current noise voltage appearing at the output end, recorded as Vn; and then the SNR may be calculated based on SNR=201 g(Vs/Vn). Or, a signal ratio may be calculated based on SNR=101 g(Ps/Pn), where Ps and Pn are effective powers of the signal and the noise, respectively.

Thirdly, for another example, when the reliability degree passes an empirical value review of one or more indexes: it may be understood that it is possible that the action amount causes a relatively large fluctuation of the sensing signal of a cardiovascular aspect, such that the sensing signal of the cardiovascular aspect becomes not meeting a specific empirical threshold requirement of the empirical value, deviating from the objective condition of the cardiovascular of the subject. Likewise, it is also possible that the action amount does not cause a relatively large fluctuation of the sensing signal of the cardiovascular aspect, such that the sensing signal of the cardiovascular aspect still meets the specific empirical threshold requirement of the empirical value, not deviating from the objective condition of the cardiovascular of the subject.

In view of the above, regardless of the sensing signal of which cardiovascular aspect, as long as it meets the reliability degree requirement, for various action amounts of the subject, the present disclosure may pre-estimate their corresponding cardiovascular characteristic reference values, store the relevant cardiovascular characteristic reference values, and accurately determine the estimation scopes corresponding to the various action amount threshold ranges. In this way, when the sensing signal of a cardiovascular aspect does not meet the reliability degree requirement, estimation of the cardiovascular characteristic parameter may be implemented by executing steps S3011 and S3012.

It needs to be noted that in conjunction with what have been described: if an estimation scope is limited by upper and lower limit parameters of data, all data within the estimation scope will be easily designed to be present as a data table, and each estimation scope corresponds to a lookup table area. It is apparent that since this embodiment may determine a corresponding cardiovascular characteristic reference value through table lookup, a first action amount of the subject and the corresponding cardiovascular characteristic reference value (e.g., heart rate, respiratory rate, oxyhemoglobin saturation, etc.) are surely pre-stored. The form of pre-storage may be a database, a datasheet, or even a text.

Another embodiment discloses how to estimate the cardiovascular characteristic parameter when the reliability degree of the sensing signal of the cardiovascular aspect satisfies the first threshold requirement. In this embodiment, step S300 specifically comprises the following sub-steps:

S3021: when the reliability degree of the sensing signal of the cardiovascular aspect satisfies the first threshold requirement, determining, based on the first estimation scope, a cardiovascular characteristic parameter baseline value corresponding to the first action amount threshold range and a cardiovascular character parameter variable corresponding to the first action amount, wherein the cardiovascular characteristic parameter baseline value is defined different from the cardiovascular character parameter variable; and

S3022: determining the estimation value of the cardiovascular characteristic parameter based on the cardiovascular character parameter variable and the cardiovascular characteristic parameter baseline value.

When the reliability degree of the sensing signal of the cardiovascular aspect satisfies the first threshold requirement, besides the cardiovascular character parameter variable corresponding to the first action amount, the cardiovascular characteristic parameter baseline value corresponding to the first action amount threshold range is additionally utilized to determine the estimation value of the cardiovascular characteristic parameter:

(1) with table lookup as an example, although both of this embodiment and the preceding embodiment determine the estimation value of the cardiovascular characteristic parameter through table lookup, the tables used in the two embodiments are not the same table:

the table in the preceding embodiment has two dimensions: the cardiovascular characteristic reference value corresponding to the first action amount, and the estimation value of the cardiovascular characteristic parameter;

the present embodiment is characterized in that the table has three dimensions: the cardiovascular character parameter variable corresponding to the first action amount, the cardiovascular characteristic parameter baseline value corresponding to the first action amount threshold range, and the estimation value of the cardiovascular characteristic parameter.

(2) it needs to be noted that the present embodiment may not only be implemented through table lookup, but also may be implemented in conjunction with a more complex approach, e.g., by computation, using a specific empirical equation, etc., which will be described in detail infra.

(3) It needs to be further noted that the cardiovascular character parameter variable corresponding to the first action amount may include a plurality of parameters, which will be described in detail infra.

(4) It needs to be still further noted that the present embodiment estimates the cardiovascular characteristic parameter only utilizing the reliability degree of the sensing signal of the cardiovascular aspect, not directly utilizing the sensing signal of the cardiovascular aspect.

In the preceding embodiment, the cardiovascular characteristic reference value is directly used as the corresponding estimation value; therefore, the cardiovascular characteristic reference value may be the pre-stored heart rate, respiratory rate, and oxyhemoglobin saturation corresponding to different action amount values;

In the present embodiment, besides estimating with the cardiovascular characteristic parameter baseline value corresponding to the first action amount threshold range, the cardiovascular character parameter variable corresponding to the first action amount may also be utilized, including the pre-stored heart rate, respiratory rate, and oxyhemoglobin saturation corresponding to different action amount values, as well as one coefficient j or a plurality of coefficients j1, j2, ji corresponding to different action amount values needed when estimating the cardiovascular characteristic parameters, and even including relevant parameters that are needed by any other estimation algorithms or empirical equations, which will be described in detail with examples. Namely, the cardiovascular characteristic parameter variables corresponding to the first action amount in the present embodiment cover a scope wider than the cardiovascular characteristic reference value corresponding to the first action amount in the preceding embodiment.

Example 1: when the subject is a human being, an example will be taken that the sensing signal of the cardiovascular aspect is a PPG signal, and the cardiovascular characteristic parameter is a heart rate:

Studies show that when a human being is performing an activity, the heart rate rises at least from a resting heart rate, and exhibits a tendency to change exponentially with increase of the action amount. When the heart rate is estimated using the exponential estimation algorithm, because the first action amount is defined in the first action amount threshold range, compared with the aforementioned that the estimation scope may be limited by upper and lower limit parameters, the estimation scope here may be an exponential data range corresponding to each action amount threshold range. The exponential data range may be limited by a plurality of specific discrete exponential values therein (e.g., with a natural constant e as the base, different powers will result in a plurality of specific discrete exponential values), or limited by a specific exponential equation corresponding to each action amount threshold range. An exemplary exponential equation is provided below:

HRₜ=HR_{b}+HRᵢ×(1−e^{α×A}^{ₜ}^{/A}^{_{b}}) (1)

In equation (1),

HRₜ denotes an estimation value of the cardiovascular characteristic parameter, here referring to an estimation value of heart rate;

Aₜ denotes to a first action amount, located in the first action amount threshold range; supposing the first action amount threshold range here is in the relatively lower level action amount range, it represents that the subject is substantially not moving, e.g., lying still or sitting still, when the interference is relatively small;

HR_{b} denotes the cardiovascular characteristic parameter baseline value corresponding to the first action amount threshold range; in view of the presumption above, the HR_{b} here represents a baseline value, which is preferably a resting heart rate, but not necessarily the resting heart rate, which will be expounded infra; the parameters HRᵢ, A_{b}, and α all corresponding to a first action amount threshold range, which are preset numerical values and may be empirical parameters or numerical vales determined by various statistics or various other predetermined approaches, corresponding to different action amount threshold ranges, wherein:

HRᵢ denotes a superimposition value of cardiovascular characteristic parameters, specifically referring to a heart rate superimposition value in the case of heart rate estimation; this value may be a positive value or a negative value, depending on HR_{b} and Aₜ, which will be expounded infra;

A_{b} denotes a baseline action amount, which is always associated with the HR_{b} and Aₜ;

α denotes a coefficient; when used for heart rate estimation, α is preferably the following empirical parameter: −3;

It may be understood that the equation above implements a method of determining an estimation value of a cardiovascular characteristic parameter (e.g., an estimation value of heart rate) by computation. In the equation above, specific values of HR_{b}, HRᵢ, A_{b}, and α may be values preset based on experience or statistics under different action amounts, e.g., they may be values associated with the resting heart rate of the subject or values associated with a long-term stable and constant heart rate value of the subject within a certain action amount range, e.g., when a certain subject is with the middle level action amount range, his/her/its heart rate is stably and constantly about 95 times per minute in a long term. In view that the specific values of HR_{b}, HRᵢ, A_{b}, and α are directly or indirectly associated with the action amount, while the first action amount corresponds to the first action amount threshold range, for the first action amount threshold range, the equation above apparently determines the estimation scope, i.e., the first action amount threshold range determines the first estimation scope.

It needs to be noted that besides the HRₜ being the estimation value that needs to be obtained and Aₜ being the first action amount that has been obtained, the values of HR_{b}, HRᵢ, A_{b}, and α may all be updated or adjusted. In other words, the cardiovascular characteristic parameter baseline value (e.g., HR_{b}), other parameters (e.g., HRᵢ, A_{b}, and α), and even broadly, respective parameters in the relevant equation, may all be updated or adjusted, as long as such adjustment makes the estimation value of the cardiovascular characteristic parameter closer to the objective condition. Updating of relevant values or parameters will be expounded infra.

It may be predicted that such updating or adjustment may vary with individuals (e.g., the updating or adjustment for athletes is always different from that for common people). It needs to be noted that because the values of HR_{b}, HRᵢ, A_{b}, and α may be updated or adjusted, as the embodiment of the present disclosure is implemented in multiple times, it is possibly unnecessary for them to be updated or adjusted any more after multiple times of updating, and each subject may obtain an estimation value of the cardiovascular characteristic parameter as accurate as possible, which may reflect the individuality of each subject. With the heart rate as an example, supposing the embodiment of the present disclosure is applied to a smart band, if the cardiovascular characteristic parameter baseline value and respective parameters in the relevant equation are constantly updated, the correspondence relationship between the action amount and the heart rate will tend to be personalized and meanwhile closer to the objective condition. It may be understood that when the HR_{b} is preferably the resting heart rate and the various values above need to be updated, it should be considered in priority to directly compute the resting heart rate with the sensing signal of the cardiovascular aspect in a lying still state so as to update the HR_{b}. If a difference (or an absolute value of the difference) between the HRₜ estimated in equation (1) above and the HR_{b} exceeds a certain threshold, it may be considered to update HRᵢ and/or A_{b}, and/or α.

With the heart rate as an example, relevant parameters will be expounded below with respect to the equation (1) above:

Although HR_{b} in the present disclosure is preferably the resting heart rate, it does not mean that the HR_{b} has to use the resting heart rate as the baseline value, which may have the following circumstances:

A. supposing the first action amount is located in the abovementioned (60, 100] low level action amount range:

Because the heart rate in the low level action amount range is higher than that in sitting still or lying still, it is appropriate that the HR_{b} uses the resting heart rate as the cardiovascular characteristic parameter baseline value corresponding to the first action amount threshold range; at this point, HRₜ is greater than HR_{b}. It is easily understood that the HRᵢ should be a positive value; at this point, A_{b} is selected as the first baseline action amount, and α is selected as a first coefficient setting value.

However, there still exists another circumstance: the (60, 100] low level action amount range is lower than the (100, 120] medium level action amount range, while the heart rate in the medium level action amount range is higher than that in the low level action amount range; therefore, HR_{b} may also adopt a certain heart rate value in the middle level action amount range (e.g., when the subject is in the middle level action amount range and his/her/its heart rate is stably and constantly around 95 times per minute in a long term, the 95 times per minute may be selected) as the cardiovascular characteristic parameter baseline value corresponding to the first action amount threshold range. At this point, because the first action amount is in the previously mentioned (60, 100] low level action amount range, HRₜ should be less than HR_{b}; it is easily understood that the HRi should be a negative value; at this point, A_{b} may select the second baseline action amount, and α may select a second coefficient setting value, wherein: the second baseline action amount has a value always different from the first baseline action amount, and the second coefficient setting value is also always different from the first coefficient setting value.

B. supposing the first action amount is located in the abovementioned (100, 120] medium level action amount range:

On one hand, because the heart rate in the medium level action amount range is higher than that when sitting still or lying still, it is appropriate for the HR_{b} to adopt the resting heart rate as the cardiovascular characteristic parameter baseline value corresponding to the first action amount threshold range; at this point, HRₜ is greater than HR_{b}. It is easily understood that the HRᵢ should be a positive value; at this point, A_{b} may select a third baseline action amount, and α may select a third coefficient setting value. Because the resting heart rate is also used as the cardiovascular characteristic parameter baseline value corresponding to the first action amount threshold range, the third baseline action amount is always identical to the value of the first baseline action amount, and the third coefficient setting value is always identical to the first coefficient setting value; however, different subjects may vary somewhat (e.g., athletes VS. common people);

On the other hand, the (100, 120] medium level action amount range is also higher than the (60, 100] low level action amount range. Because the heart rate in the medium level action amount range is higher than that in the low level action amount range, the HR_{b} also adopts a certain heart rate value in the low level action amount range as the cardiovascular characteristic parameter baseline value corresponding to the first action amount threshold range; at this point, because the first action amount is in the aforementioned (100, 120] medium level action amount range, HRₜ should be greater than HR_{b}. It is easily understood that the HRᵢ should still be a positive value. A_{b} selects a fourth baseline action amount, and α selects a fourth coefficient setting value, wherein the fourth baseline action amount is always different from the value of the third baseline action amount, and the fourth coefficient setting value is also always different from the third coefficient setting value.

In view of the above, besides the HRₜ being an estimation value that needs to be obtained and Aₜ being the first action amount that has been obtained, the values of HR_{b}, HRᵢ, A_{b}, and α may all be updated and adjusted, as long as such adjustment causes the estimation value of the cardiovascular characteristic parameter closer to the objective condition.

It may be understood that the HR_{b} is preferably a resting heart rate, and when it is needed to update the respective values above, it should be considered to first update the HR_{b}: when the subject is in a sitting still or lying still state, the resting heart rate is directly computed with the sensing signal of the cardiovascular aspect so as to update the HR_{b}; the computing method may refer to the prior art or other approaches disclosed infra. More preferably, when the subject is sitting still or lying still, and the reliability degree (confidence or other index of reliability degree) of the sensing signal of the cardiovascular aspect meets the aforementioned requirements, update the HR_{b}.

It needs to be noted that when the HR_{b} is set, α is −3, and the actual heart rate of the subject under any action amount has been obtained through other measuring instrument, in order to preset the HRᵢ and A_{b}, the HRᵢ may be set first and then the A_{b}; or the A_{b} may be set first and then the HRᵢ. In this case, the HRᵢ and A_{b} may be updated using various kinds of appropriate fitting methods.

It needs to be further explained that although the aforementioned example 1 discloses an embodiment of estimating the cardiovascular characteristic parameter of heart rate using the non-linear relationship (exponential relationship) represented by equation (1), it does not mean that the present disclosure cannot estimate a cardiovascular characteristic parameter using a linear relationship. It may be understood that if enough action amount threshold ranges and estimation scopes are partitioned within the scope from the subject's lowest extreme action amount to the highest extreme action amount and absolute values of a difference between the upper and lower limits of each action amount threshold range and a difference between the upper and lower limits of the corresponding estimation scope are within a small enough numerical value, those skilled in the art may perform linear fitting within each estimation scope to thereby estimate the cardiovascular characteristic parameter irrespective of the each estimation scope being defined by the parameters of which non-linear relationship and its equation, and irrespective of the sensing signal of the cardiovascular aspect being a PPG signal or an ECG signal. As to the differences between different equations, they are dependent on the preference of estimation accuracy or efficiency.

Example 2: when the subject is a person, an example is taken that the sensing signal of the cardiovascular aspect is a PPG signal and the cardiovascular characteristic parameter is a respiratory rate or an oxyhemoglobin saturation:

The subject's blood circulation is associated with his/her action amount, while the heart rate, the respiratory rate, the oxyhemoglobin saturation, and even the concentration of a certain substance in the blood may be estimated through the sensing signal of the cardiovascular aspect of the blood. Naturally, the action amount is associated with the respiratory rate, the oxyhemoglobin saturation, and even the concentration of a certain substance. The aforementioned example 1 is directed to the heart rate, while the present example 2 is directed to a respiratory rate or the oxyhemoglobin saturation. Additionally, the first action amount still takes the acceleration parameter as an example. It may be understood that changes of the velocity parameter may determine the acceleration parameter, while the velocity parameter may be determined based on the pace count and the pace frequency parameter in conjunction with a distance parameter.

In statistics, the Poisson correlation coefficient is for reflecting a correlation relationship between variables. The correlation coefficient between two variables X, Y may be represented by ρ_{XY}, which may be calculated through the equation below:

$${ρ}_{XY} = \frac{cov ( X , Y )}{{σ}_{X} {σ}_{Y} } \qquad ( 2 )$$

In equation (2):

σ_{X} denotes a square deviation of X, σ_{Y} denotes a square deviation of Y, and cov(X, Y) denotes a co-variance of variable X and variable Y.

The correlation coefficient ρ_{XY} is evaluated between −1 and 1; |ρ_{XY}| is evaluated between 0 and 1; the larger the value of |ρ_{XY}| is, the larger the change of Y incurred by the change of X is;

when ρ_{XY} is 0, X, Y are not correlated; when ρ_{XY} is greater than 0, X, Y are positively correlated; when ρ_{XY} is smaller than 0, X, Y are inversely correlated;

when |ρ_{XY}| is 1, X, Y are linearly correlated;

when |ρ_{XY}| is smaller than 1, change of X incurs partial change of Y;

when |ρ_{XY}| is smaller than 0.3 but not 0, X and Y may be regarded as low correlation; when |ρ_{XY}| is larger than 0.8 but smaller than 1, X and Y may be regarded as high correlation; when |ρ_{XY}| is other case, X and Y may be regarded as medium correlation.

Therefore, the present disclosure may pre-partition enough action amount threshold ranges within the scope from the lowest extreme action amount to the highest extreme action amount of the subject, and measure the specific action amount and the specific respiratory rate or oxyhemoglobin saturation within each action amount threshold range, then: within the each action amount threshold range, a correlation coefficient β between the mounting amount and the respiratory rate or the oxyhemoglobin saturation using the equation (2). Meanwhile, the respiratory rate or oxyhemoglobin saturation of the subject when sitting still or lying still is pre-obtained, used as the respiratory rate base value or the oxyhemoglobin saturation base value. Then, the respiratory rate or the oxyhemoglobin saturation at the first action amount is estimated using the equation (3):

BRₜ=BR_{b}+BRᵢ×βᵢ (3)

In the equation (3), an example is taken that the BRₜ is the estimation value of the respiratory rate;

BR_{b} is the respiratory rate baseline value corresponding to the first action amount threshold range; in view of the presumption above, because the action amount when lying still or sitting still is relatively very low and the interference factors are few, the baseline value represented by BR_{b} is preferably the respiratory rate when lying still or sitting still, but is not necessarily the respiratory rate when lying still or sitting still, which will be expounded infra;

βᵢ is the correlation coefficient between the action amount and the respiratory rate within the first action amount threshold range; βᵢ is evaluated between −1 and 1; as mentioned above, βᵢ may be obtained through the equation (2);

BRᵢ is a superimposition value of respiratory rates; BRᵢ may be an empirical parameter or a numerical value determined by various other pre-determining approaches, wherein: when the BR_{b} uses the respiratory rate when lying still or sitting still as a baseline value, if the action amount is a motion state, e.g., striding or running, then BRᵢ×βᵢ is a positive value. It needs to be noted that when βᵢ is obtained through the equation (2), the specific numerical value of βᵢ and its positive or negative have been determined, and the positive or negative of BRᵢ×βᵢ is decided by βᵢ.

It may be understood that for each action amount threshold range, the equation implements a method of determining an estimation value of the cardiovascular characteristic parameter (e.g., the estimation value of the respiratory rate) by linear fitting. In the equation, BR_{b} is the cardiovascular character parameter baseline value in the embodiment, while BRᵢ and βᵢ are the cardiovascular characteristic parameter variables in the embodiments or coefficients in an equation in a broader sense. At to the specific values of BR_{b}, BRᵢ, and βᵢ, they may be values preset according to experience or statistics under different action amounts, e.g., they may be values associated with the respiratory rate of the subject when sitting still, or may be values associated with a long-term stable and constant respiratory rate of the subject within a certain action amount range. For example, when a certain subject is in the medium level action amount range, its respiratory rate is stable and constantly around 22 times per minute in a long term. In view that the specific values of the BR_{b}, BRᵢ, and βᵢ in the equation are directly or indirectly associated with the action amount, while the first action amount corresponds to the first action amount threshold range, for the first action amount threshold, the above equation apparently determines the estimation scope, i.e., the first action amount threshold range determines the first estimation scope.

It needs to be noted that besides the BRₜ being the estimation value that needs to be obtained, the values of BR_{b}, BRᵢ, and βᵢ may all be updated or adjusted, as long as such adjustment causes the estimation value of the cardiovascular characteristic parameter closer to the objective condition. Updating of relevant values or parameters will be expounded infra.

It may be predicted that such updating or adjustment may vary with individuals (e.g., the updating or adjustment for athletes is always different from that for common people). It needs to be noted that because the values of BR_{b}, BRᵢ, and βᵢ may be updated or adjusted, as the embodiment of the present disclosure is implemented in multiple times, it is possibly unnecessary for them to be updated or adjusted any more after multiple times of updating, and each subject may obtain an estimation value of the cardiovascular characteristic parameter as accurate as possible, which may reflect the individuality of each subject. With the respiratory rate as an example, supposing the embodiment of the present disclosure is applied to a smart band, if the cardiovascular characteristic parameter baseline value and the cardiovascular characteristic reference value corresponding to the first action amount are constantly updated, the correspondence relationship between the action amount and the respiratory rate will tend to be personalized and meanwhile closer to the objective condition. It may be understood that when the HR_{b} is preferably the respiratory rate when sitting still or lying still and the respective values need to be updated, it should be considered in priority to directly compute the resporatory rate with the sensing signal of the cardiovascular aspect in a lying still or sitting still state so as to update the BR_{b}. The computing method may refer to the prior art, and the present disclosure does not attempt to propose a new approach.

If a difference (or an absolute value of the difference) between BRₜ and BR_{b} estimated according to the equation (3) exceeds a certain threshold, it may be considered to update the BRᵢ and/or use equation (2) to compute βᵢ. Under this circumstance, the BRᵢ may be updated using any appropriate fitting method.

It needs to be noted that although the aforementioned example 2 discloses an embodiment of estimating the cardiovascular characteristic parameter of respiratory rate using the linear relationship represented by equation (3) when the sensing signal of the cardiovascular aspect is a PPG signal, it does not mean that the present disclosure cannot estimate the respiratory rate using a non-linear relationship. It may be understood that if enough action amount threshold ranges and estimation scopes are partitioned within the scope from the subject's lowest extreme action amount to the highest extreme action amount and absolute values of a difference between the upper and lower limits of each action amount threshold range and a difference between the upper and lower limits of the corresponding estimation scope are within a small enough numerical value, those skilled in the art may perform linear fitting or nonlinear fitting within each estimation scope to thereby estimate the cardiovascular characteristic parameter irrespective of the sensing signal of the cardiovascular aspect being a PPG signal or an ECG signal. As to the differences between different equations, they are dependent on the preference of estimation accuracy or efficiency.

It may be understood that as far as fitting is concerned, the example 2 is characterized in that: on one hand, how to resolve the correlation coefficient and linear fitting; on the other hand, the process of linearly fitting the equation is directly computing the relevant estimation value only using the baseline value, the superimposition value and the correlation coefficient (wherein the superimposition value and the correlation coefficient are a parameter variable and a coefficient in an equation of a broader sense), without utilizing the sensing signal in the cardiovascular aspect. Naturally, the process is irrelevant to the type of the sensing signal of the cardiovascular aspect and the type of the estimated cardiovascular characteristic parameter. Therefore, it is apparent that the example 2 is still applicable even utilizing the PPG signal to estimate the oxyhemoglobin saturation or other cardiovascular characteristic parameter as mentioned above.

In addition, even the sensing signal of the cardiovascular aspect is an ECG signal, similar to the example 2, the present disclosure may also be easily applied to estimate the oxyhemoglobin saturation or respiratory rate or other cardiovascular characteristic parameter as mentioned above. In view of the above, the present disclosure will not elaborate the embodiments of the oxyhemoglobin saturation or other cardiovascular characteristic parameters through the example 3 and relevant equations.

Furthermore, by extending the example 1 and the example 2, if enough action amount threshold ranges and estimation scopes are partitioned within the scope from the subject's lowest extreme action amount to the highest extreme action amount and absolute values of a difference between the upper and lower limits of each action amount threshold range and a difference between the upper and lower limits of the corresponding estimation scope are within a small enough numerical value, the present disclosure may also perform the following linear fitting or non-linear fitting within each estimation scope: forming the equation within each estimation scope by performing a unary linear regression or multiple linear regression or even multiple curve regression using numerical calculation software such as MATLAB or statistical analysis software such as SPSS, to estimate cardiovascular characteristic parameters such as heart rate, respiratory rate, oxyhemoglobin saturation, and etc.; for example, forming an equation through the multiple linear regression or multiple curve regression so as to estimate the heart rate variability (HRV) using a PPG signal or estimate the respiratory rate using an ECG signal. It may be understood that the correlation coefficient and linear regression or nonlinear regression are also applicable to the embodiments infra.

It needs to be noted that the absolute value of the difference between the upper limit and the lower limit of the estimation scope may be limited through corresponding thresholds, thereby limiting how to partition the estimation scope and further limiting how to partition the action amount threshold range.

Likewise, similar to Example 2, when forming other equations through the multiple linear regression or multiple curve regression, particularly used for estimating the respiratory rate/oxyhemoglobin saturation, the oxyhemoglobin saturation/respiratory rate may be additionally introduced as one element in the multiple elements, in addition to the action amount and the relevant baseline value. This is because the respiratory rate and the oxyhemoglobin saturation have a correlation; besides, the respiratory rate and the blood pressure instant variability (BPIV) also have a correction, such that it may also be considered to additionally introduce the BPIV as another element in the multiple element. In contrast, the oxyhemoglobin saturation/respiratory rate is always not needed to be introduced when estimating the heart rate, because the prior art has provided a considerable number of algorithms for estimating the heart rate; however, theoretically, the oxyhemoglobin saturation/respiratory rate may be introduced.

Similar to example 2, when forming an equation through the multiple linear regression or multiple curve regression so as to estimate the heart rate variability (HRV) using a PPG signal, the respiratory rate may be additionally introduced as one element in the multiple elements, in addition to the action amount and the relevant baseline value. This is because the HRV has a correlation with the respiratory rate. More particularly, the blood pressure instant variability (BPIV) may be further introduced as another element in the multiple elements. This is because the respiratory rate and the BPIV also have a correlation. It needs to be noted that for estimating the HRV or the BPIV, an ECG signal may be additionally introduced in addition to the PPG signal to perform the multiple linear regression or multiple curve regression, so as to enhance the accuracy. Even the individual values of the estimated HRV or BPIV are not objective enough, continuous estimations of the HRV or BPIV are significant, because their change tendencies may provide a guidance or warning to health.

Based on the above embodiments, particularly examples 1 and 2, and more broadly, in another embodiment, step S300 specifically comprises the following sub-steps:

S3031: when the reliability degree of the sensing signal of the cardiovascular aspect satisfies a second threshold requirement, determining a computation equation corresponding to the first estimation scope and respective parameters of the equation, wherein at least one parameter of the equation corresponds to the first action amount;

S3032: determining the estimation value of the cardiovascular characteristic parameter based on the computation equation.

In this embodiment, there may include one coefficient k or a plurality of coefficients k₁, k₂, kᵢ, which correspond to different action amount values and are needed for estimating the cardiovascular characteristic parameter, or may even include relevant parameters needed by any other estimation algorithm or empirical equations, wherein the specific empirical equations and equation parameters, etc. The second threshold requirement may be identical to or different from the abovementioned first threshold requirement.

It needs to be noted that the parameters of the equation may include a plurality of parameters, as long as they may be used to determine the estimation values of the cardiovascular characteristic parameters. However, regardless of the present embodiment or the above Examples 1, 2, at least one parameter corresponds to the first action amount. As such, the embodiment may compute the estimation value of the cardiovascular characteristic parameter not directly using the sensing signal of the cardiovascular aspect, but in conjunction with the first action amount and the relevant equation.

In another embodiment, step S300 specifically comprises the following sub-steps:

S3041: determining a cardiovascular characteristic reference value corresponding to the first action amount based on the first estimation scope when a change ratio of the first action amount within a second time interval to the preceding action amount is within a third threshold range;

S3042: using the cardiovascular characteristic reference value corresponding to the first action amount as the estimation value of the cardiovascular characteristic parameter.

The present embodiment is different from the previous embodiments. Based on the first embodiment, the present embodiment additionally introduces a change ratio of the first action amount within the second time interval to the preceding action amount. This is still originated from the action amount of the subject. It may be understood that if the action amount almost has no significant changes within a certain period of time, the estimation frequency and computational capacity may be reduced, and in special circumstances, it is even unnecessary to cause relevant sensors for sensing signals of the cardiovascular aspect to work, especially when the subject's cardiovascular is in a healthy level. The present embodiment is just for energy conservation in this circumstance: it may be understood, execution of steps S3041 and S3042 means the present embodiment does not perform any narrow-sense estimation computation, but determining the cardiovascular characteristic reference value corresponding to the first action amount directly based on the first estimation scope, so as to directly use it as the estimation value of the cardiovascular characteristic parameter under the current action amount. It needs to be noted that because the action amount almost has no significant change, the cardiovascular characteristic reference value corresponding to the first action amount under this circumstance may also be the previously determined cardiovascular characteristic parameter estimation value or may be determined based on one or more historical estimation values of the cardiovascular characteristic parameter, e.g., the preceding historical value, or an arithmetic mean value of previous multiple historical values, or any appropriate determining method corresponding to the estimation scope based on one or more historical values (e.g., resolving the cardiovascular characteristic parameter estimation value based on historical values in conjunction with corresponding linear or nonlinear fitting equations).

In conjunction with the disclosure in the abovementioned first embodiment, if all data within the estimation scopes are existent as data tables, each estimation scope corresponds to a lookup table area; apparently, now that the present embodiment may determine the corresponding cardiovascular characteristic parameter, the first action amount of the subject and the corresponding cardiovascular characteristic parameter (e.g., the heart rate, the respiratory rate, the oxyhemoglobin saturation) may be pre-stored. The form of pre-storage may be a database, a data sheet, or even a text.

In another embodiment, step S300 specifically comprises the following sub-steps:

S3051: when a change ratio of the first action amount within a second time interval to the preceding action amount is within a fourth threshold range, determining a computation equation corresponding to the first estimation scope and respective parameters of the equation, wherein at least one parameter of the equation corresponds to the first action amount;

S3052: determining the estimation value of the cardiovascular characteristic parameter based on the computation equation.

Corresponding to the preceding embodiment, when the change ratio of the first action amount within a second time interval to the preceding action amount is within a fourth threshold range, the present embodiment addresses the circumstance when the sensing signal of the cardiovascular aspect changes significantly.

It needs to be noted that the parameters of the equation may include a variety of parameters, as long as they may be used to determine the estimation value of the cardiovascular characteristic parameter. Similar to examples 1 and 2, the parameters of the equation may be one or more baseline values, or superimposition values, or coefficients, or parameters of an equation involved in other estimation algorithms, which corresponding to different action amount values.

It needs to be further noted that the present embodiment may compute the estimation value of the cardiovascular characteristic parameter not directly using the sensing signal of the cardiovascular aspect, e.g., examples 1 and 2 as previously mentioned.

In a further embodiment, the step S300 specifically comprises the following sub-steps:

S3061: when an absolute value of a difference between a largest value and a minimum value of the sensing signal of the cardiovascular aspect within the second time interval is within a fifth threshold range, determining a cardiovascular characteristic reference value corresponding to the first action amount based on the first estimation scope;

S3062: using the cardiovascular characteristic reference value corresponding to the first action amount as an estimation value of the cardiovascular characteristic parameter.

The present embodiment is different from the previous embodiments. Based on the first embodiment, the present embodiment additionally introduces the absolute value of a difference between a largest value and a minimum value of the sensing signal of the cardiovascular aspect within the second time interval. In other words, it additionally introduces a change degree of the sensing signa of the cardiovascular aspect. This is still originated from the action amount of the subject.

Exemplarily and non-limiting, if the action amount almost has no significant changes within a certain period of time, the estimation frequency may be reduced, and in special circumstances, it is even unnecessary to cause relevant sensors for sensing signals of the cardiovascular aspect to work, especially when the subject's cardiovascular is in a healthy level. the present embodiment is just for energy conservation in this circumstance: it may be understood, execution of steps S3061 and S3062 means the present embodiment does not perform any narrow-sense estimation computation, but determining the cardiovascular characteristic reference value corresponding to the first action amount directly based on the first estimation scope, so as to directly use it as the estimation value of the cardiovascular characteristic parameter under the current action amount.

In this exemplary circumstance, because the action amount almost has no significant change, the cardiovascular characteristic reference value corresponding to the first action amount under this circumstance may also be the previously determined cardiovascular characteristic parameter estimation value or may be determined based on more historical estimation values of the cardiovascular characteristic parameter, as discussed above.

In conjunction with the disclosure in the abovementioned first embodiment, if all data within the estimation scopes are existent as data tables, each estimation scope corresponds to a lookup table area; apparently, now that the present embodiment may determine the corresponding cardiovascular characteristic parameter, the first action amount of the subject and the corresponding cardiovascular characteristic parameter (e.g., the heart rate, the respiratory rate, the oxyhemoglobin saturation) may be pre-stored. The form of pre-storage may be a database, a data sheet, or even a text.

In another embodiment, step S300 specifically comprises the following sub-steps:

S3071: when an absolute value of a difference between a largest value and a minimum value of the sensing signal of the cardiovascular aspect within the second time interval is within a sixth threshold range, determining a computation equation corresponding to the first estimation scope and respective parameters of the equation, wherein at least one parameter of the equation corresponds to the first action amount;

S3072: determining the estimation value of the cardiovascular characteristic parameter based on the calculation equation.

Corresponding to the preceding embodiment, when the absolute value of a difference between a largest value and a minimum value of the sensing signal of the cardiovascular aspect within the second time interval is within a sixth threshold range, the present embodiment addresses the circumstance when the sensing signal of the cardiovascular aspect changes significantly but between the normal largest value and minimum value.

It needs to be noted that the parameters of the equation may include a variety of parameters, as long as they may be used to determine the estimation value of the cardiovascular characteristic parameter. Similar to examples 1 and 2, the parameters of the equation may be one or more baseline values, or superimposition values, or coefficients, or parameters of an equation involved in other estimation algorithms, which corresponding to different action amount values.

It needs to be further noted that the present embodiment may compute the estimation value of the cardiovascular characteristic parameter not directly using the sensing signal of the cardiovascular aspect, e.g., examples 1 and 2 as previously mentioned.

In a further embodiment, the upper limit and/or lower limit of the first estimation scope have an attribute of varying with the first action amount.

As discussed above, the action amount corresponds to a corresponding estimation scope. Therefore, it may be understood that such a circumstance exists: the upper limit and/or lower limit of the first estimation scope have an attribute of varying with the first action amount.

In another embodiment, data in the first estimation scope has an attribute of updating with the estimation value of the cardiovascular characteristic parameter.

It may be understood that when the reliability degree of the sensing signal of the cardiovascular aspect meets the threshold requirement, each calculated estimation value of the cardiovascular characteristic parameter will be closer to the objective condition; then such a circumstance exists: with table lookup as an example, supposing the data directly determined in the first estimation scope are different from the current estimation value of the cardiovascular characteristic parameter, it may be considered to update the data in the first estimation scope using the estimation value of the cardiovascular characteristic parameter. It may be understood that the data in the first estimation scope may be the data addressed in the aforementioned table lookup, or may be the data of relevant parameters in the aforementioned respective equations, i.e., the setting values of the various variables, and these values may be updated or adjusted.

In another embodiment, the method further comprises the following steps:

S400: classifying the estimation value of the cardiovascular characteristic parameter.

In the embodiment, it may be understood that if the action amount threshold range corresponding to sitting still or lying still corresponds to the (40, 60] relatively lower level action amount range, the reliability degree of the sensing signal of the cardiovascular aspect under the relatively lower action amount range is regarded as a first high reliability level, exemplarily denoted as 1.0; the reliability degree of the sensing signal of the cardiovascular aspect under the (60, 100] low level action amount range is regarded as a second high reliability level, exemplarily denoted as 0.8; the reliability degree of the sensing signal of the cardiovascular aspect under the (100, 1201 medium level action amount range is regarded as a third high reliability level, exemplarily denoted as 0.7. Then, to reflect more information, the estimation value of the cardiovascular characteristic parameter may be classified. One of the classification principles may be the first high reliability level, the second high reliability level, and the third high reliability level.

In the other embodiment, the method further comprises the following steps:

S500: when a change ratio of multiple historical data of the estimation value of the cardiovascular characteristic parameter is within a seventh threshold range, determining the estimation value of the cardiovascular characteristic parameter every the third time interval at least based on the preceding historical data of the estimation value of the cardiovascular characteristic parameter; wherein the sensing signal of the cardiovascular aspect is not obtained at the third time interval period.

The present embodiment has the following characteristics: regardless of which action amount threshold range the action amount is specifically in, as long as the change ratio of the multiple historical data of the previously determined cardiovascular characteristic parameter estimation value is within the seventh threshold range, particularly when the seventh threshold range represents that the multiple historical data have no significant change, the present disclosure may further save energy based on the estimation values of the cardiovascular characteristic parameters that have been previously determined, and it is only needed to determine the latest estimation value of the cardiovascular characteristic parameter once every the third time interval; the present embodiment is particularly suitable for estimating the cardiovascular characteristic parameter of the subject without cardiovascular diseases when sleeping.

Moreover, at this point, the estimation value of the cardiovascular characteristic parameter is determined at least based on a preceding historical data of the estimation value of the cardiovascular characteristic parameter: not only one previous historical data may be used as the estimation value of the cardiovascular characteristic parameter that needs to be determined, but also an average value of the previous two or N historical data may be used as the estimation value of the cardiovascular characteristic parameter that needs to be determined. It may be understood that other data fitting methods are also applicable.

In another embodiment, the method further comprises a step of:

S600: estimating a time interval of obtaining the sensing signal of the cardiovascular aspect at the next time based on the estimation value of the cardiovascular characteristic parameter.

It may be understood that within a sensing period of the sensing signal of the cardiovascular aspect, the cardiovascular characteristic parameter is substantively a waveform, while this waveform has a peak and a cycle. With a health subject as an example, its waveform is periodical; therefore, based on the estimation value of the cardiovascular characteristic parameter (which may be one estimation value or multiple historical data), it may be predicted that only after a certain time interval, will the sensing signal of the cardiovascular aspect have the peak and/or periodical information that is needed for estimating the cardiovascular characteristic parameter. Therefore, in this sense, it is meaningful to estimate the time interval of obtaining the sensing signal of the cardiovascular aspect at the next time, particularly when enough historical data have been accumulated.

In another embodiment, the method further comprises the following step:

S700: estimating the next time of switching on or switching off the following sensor based on the estimation value of the cardiovascular characteristic parameter: the sensor for obtaining the sensing signal of the cardiovascular aspect.

With reference to the preceding embodiment, it may be understood that estimating the next time of switching on or switching off the sensor may facilitate further reduction of energy consumption. Therefore, the sensor for obtaining the sensing signal of the cardiovascular aspect is switched off when it is not needed, and switched on when it is needed.

In another embodiment, the method further comprises the following step:

S800: when a difference (or an absolute value of the difference) between the estimation of the cardiovascular characteristic parameter and the cardiovascular characteristic parameter baseline value corresponding to the first action amount threshold range is greater than an eighth threshold, calibrating the cardiovascular characteristic parameter baseline value corresponding to the first action amount threshold range using the estimation value of the cardiovascular characteristic parameter, causing the absolute value of the difference between the estimation of the cardiovascular characteristic parameter and the cardiovascular characteristic parameter baseline value corresponding to the first action amount threshold range not greater than the eighth threshold.

As previously mentioned, the cardiovascular characteristic parameter baseline value corresponding to the first action amount threshold range may be updated or adjusted and have an attribute of being updated with the estimation value of the cardiovascular characteristic parameter. The present embodiment describes a policy of calibrating to update the cardiovascular characteristic parameter baseline value corresponding to the first action amount threshold range. It needs to be noted again that the updating is making the estimation value closer to the objective condition.

In addition, in conjunction with the description above, the updating may be directed to any numerical value related to the upper and lower limits of the estimation scope and any parameters of equations related to the estimation scope. In other words, the updating is directed to the estimation scope. This is because the estimation scope is either defined by numerical values or defined by the equations. Therefore, updating the estimation scope is just for being closer to the objective condition and implementing an individualized solution for the subject. It may be understood that any numerical value related to the upper and lower limits of the action amount threshold range may also be updated, as long as facilitating the multiple linear regression or multiple curve regression mentioned above.

As described in the previous embodiments, relevant embodiments may compute the cardiovascular characteristic parameters not directly utilizing the sensing signal of the cardiovascular aspect, but utilizing the reliability degree of the sensing signal of the cardiovascular aspect, relevant baseline value, superimposition value and other coefficients, or relevant equations in a broader sense.

Relevant embodiments below address how to determine an estimation value of a heart rate directly using the sensing signal of the cardiovascular aspect when the sensing signal of the cardiovascular aspect is a PPG signal.

Hereinafter, other embodiments will be expounded with reference to the accompanying drawings.

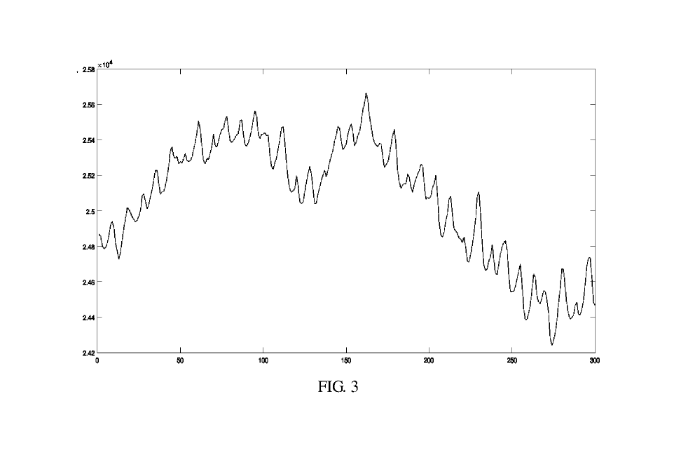

Refer to FIG. 2 showing a waveform diagram of an original PPG (photoplethysmography) in a static state and FIG. 3 showing a waveform diagram of an original PPG (photoplethysmography) in a dynamic state, wherein the horizontal axis corresponds to time, and the longitudinal axis corresponds to amplitude. The numerical values in the horizontal axis are sample points. The translation relationship between sample points and time is that with t0 as the point 0 of the horizontal axis, the sample point n in the horizontal axis is the n+1^{st} sample point starting from time t0 as the first sample point; in the case of sampling at a frequency of 25 Hz, the time corresponding to the sample point n is n/25 s; for example, the time corresponding to 300 in the horizontal axis in FIG. 3 is 12 s.

As previously mentioned, when analyzing the acceleration data, changes of the acceleration may also be used as one of the grounds for assessing human body activities. For example, if the change amount accumulation and computation is made to every 25 pieces of acceleration data, and the sampling rate of the sensor is set to 25 Hz (i.e., acquiring 25 pieces of data in 1 s), the scope of the sample value of each sample data is [0, 255], and the corresponding acceleration scope is [0, 2 g], where g denotes a gravitational acceleration. By summing the absolute values of the differences between two adjacent pieces of sampling data within every 1 second, an accumulation value a of the differences of the acceleration sampling values for 1 s is derived; by resolving an average value A of the values a in each 10 seconds, the value A is the action amount in a unit time.

When A≤M, it is believed as a static state;

When A>M, it is believed as a dynamic state;

M may be used as a threshold for differentiating dynamic state from static state. In the present disclosure, the action amount threshold is an empirical value, supposed to be 60; then the corresponding acceleration value is 4.6 m/s2.

Based on the motion data analysis, motion frequency (e.g., pace frequency) may be further obtained, or activity types may be differentiated, e.g., differentiating low-frequency activities and high-frequency activities in a dynamic state or low-intensity activities from high-intensity activities, e.g., walking and running.

Likewise, a sleep depth in a sleep state is determined based on the motion data (e.g., value A). When a person is in a sleep state, its limbs are not completely static. For example, the body and limbs will generate a certain degree of activity, i.e., the action amount within a unit time is within a certain scope. The sleep depth is divided into 5 levels from light to heavy, i.e., 0 degree, 1^{st} degree, 2^{nd} degree, 3^{rd} degree, and 4^{th} degree. The larger the degree is, the smaller the continuous action amount is, and the deeper the sleep is. The default sleep depth is 0 degree, exemplarily but not restrictively:

When A≤40, it may be defined as a sleep state;

When 30<A≤40, it may be defined as 0 degree state of the sleep, i.e., an initial sleep state;

When 25<A≤30, it may be defined as 1^{st} degree state of the sleep;

When 20<A≤25, it may be defined as 2^{nd} degree state of the sleep;

When 10<A≤20, it may be defined as 3^{rd} degree state of the sleep;

When 0<A≤10, it may be defined as 4^{th} degree state of the sleep, which may be believed as the deepest sleep, when the action amount is almost 0.

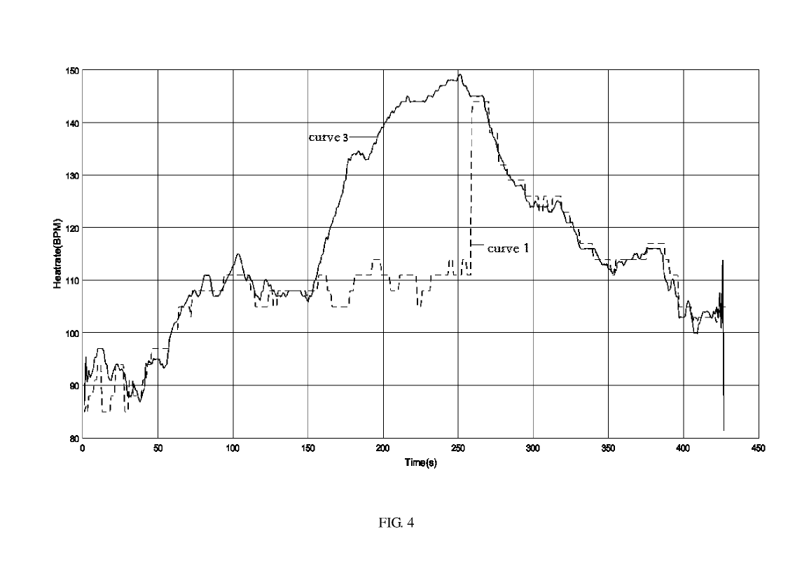

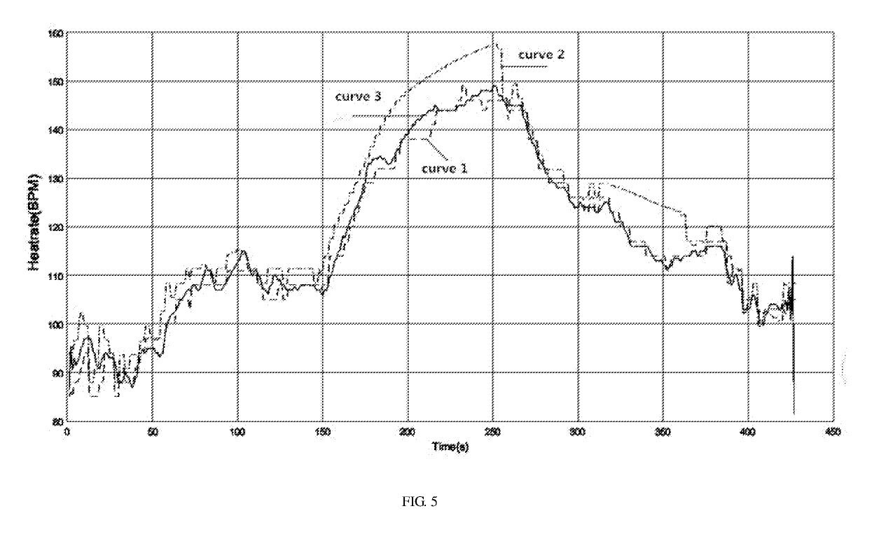

FIGS. 4 and 5 are all hear rate data diagrams in a dynamic state (during running), where the original reference heart rate value and the updated reference heart rate value are compared with the actual heart rate value. Curve 1 in FIG. 4 represents heart rate search values obtained with reference to an existing heart rate search technology, which are used as reference heart rate values; Curve 3 in FIGS. 4 and 5 represents actual heart rate values; Curve 2 in FIG. 5 represents heart rate prediction values based on a fitting equation of the action amount to heart rates; and some reference heart rate values of Curve 1 in FIG. 5 have been calibrated based on the fitting equation of the action amount to the heart rates.

The actual heart rates are measured by POLAR Heart Rate Monitor (Polar H7 Bluetooth Heart Rate Sensor) which is recognized to be more accurate in measuring sports heart rate. As shown in FIG. 5, during exercising, the original reference heart rate value is largely different from the actual heart rate value at 160˜260 s (the largest difference is about 40 times per minute). After adjusting the reference heart rate value using a fitting equation, e.g., during the 160˜260 s range in FIG. 5, the adjusted reference heart rate value is closer to the actual heart rate value than the original reference heart rate value in FIG. 4.

At each time of obtaining the reference heart rate value, a heart rate signal quality indicator (SI) and a heart rate effective indicator (AI) may be obtained as well.

As above mentioned, with the heart rate as an example, the present disclosure may reduce the computation capacity and power consumption and further calibrate the algorithm based on the differences of individual users. For different users, with increase of the time of their using the device, the measured heart rates will generally become more and more accurate. For different users, the heart rate measurement of each user will be closer to the actual value.

In one embodiment, each sleep depth and a lower limit of the estimation scope corresponding to its action amount may be set according to the lower limit heart rate;

The lower limit heart rate is computed through the following equation:

$$Hr\_lim = \frac{{\sum }_{j = 1}^{Date} {Hr\_sleep}_{j}}{Date} ,$$

In the equation:

Hr_lim denotes a lower-limit heart rate; Date denotes the number of days of continuous measurement; Hr_sleepⱼ denotes a heart rate value when the sleep depth is measured to be 4^{th} degree at the j^{th} day.

Changes of the heart rates of a person within 24 hours a day have a certain regularity. The heart rate is relatively low when the person is still or quiet; while the heart rate rises when the person is active; the larger the active amount is, the higher the heart rate level is; when the active amount is maintained at a certain level, the heart rate will be maintained within a relatively stable scope. For example, when sleeping at night and waking up in the morning, the body of a human being is in a basal metabolic state, and the frequency of heart beat is at a relatively lower level; with activities after getting up, the frequency of heart beat will be maintained at a higher level.

The resting heart rate is also referred to as a silent heart rate, which refers to the times of heart times per minute in a sober, inactive, and silent state. For a common person, the scope of resting heart rate is between 40˜100 times per minute. If the user is sober at certain time T, and within continuous 15 minutes before T, 40<A≤60, the heart rate measured at time T is the resting heart rate.

In a static state with a relatively deep sleep degree, the heart rate of a human being is relatively low. When the sleep depth is the 4^{th} degrees, the measured heart rate may be used as a lower limit heart rate. The present disclosure resolves an average value of measurement values of every day within a certain continuous number of days as the lower limit heart rate of the user within that period of time.

The lower limit heart rate varies with individuals, and sometimes the difference may be very large. For example, the lower limit heart rate of an athlete may be around 40 times per minute, while the lower limit heart rate of some common people is around 75 times per minute; the latter is nearly twice of the former. The average heart rate or highest heart rate may also vary greatly with individuals. With the same action amount or motion frequency, the heart rates also differ. Considering the heart rate differences between individuals, individual parameters may be set, or the parameters may be adjusted according to individual differences, so as to optimize the algorithm. Besides, after obtaining the lower limit heart rate, the lower limit of the estimation scope corresponding to each sleep depth may be set in a static state.

In one embodiment, step S300 specifically comprises:

S3081: when the sensing signal of the cardiovascular aspect is a PPG signal and the cardiovascular characteristic parameter is a heart rate, computing the heart rate directly using the PPG signal and using the calculated heart rate as a reference heart rate value;

Exemplarily, for the PPG signal, the heart rate may be computed using any prior art to obtain the reference heart rate value, for example: firstly processing the original waveform of the PPG signal to obtain an original time-domain AC signal; further filtering and then performing Fourier transformation, the sample signal segments are subjected to statistics and analysis within the frequency domain; within the search scope, a frequency value corresponding to the largest peak in the frequency domain is taken, thereby obtaining a heart rate that is used as a reference heart rate value;

S3082: dividing an amplitude of a frequency point value in a spectrogram corresponding to the reference heart rate by the sum of all frequency point amplitudes, a resulting ratio being used as the heart rate signal quality indicator;

S3083: when the heart rate signal quality indicator satisfies a requirement, determining a motion frequency based on the first action amount;

S3084: performing adaptive filtering to the PPG signal using the motion frequency;

S3085: computing the heart rate based on the filtered signal, used as a transition value of the heart rate;

as to how to compute the heart rate based on the filtered signal, a frequency value corresponding to its largest peak may be taken as the transition value of the heart rate;

S3086: determining a gain of Kalman filtering based on a heart rate effective indicator corresponding to the first action amount;

The heart rate effective indicator (AI) has a correspondence relationship with the first action amount. The first action amount may correspond to sitting still or lying still, or may correspond to a certain motion. For example, based on the empirical value, preferably, the heart rate effective indicator is evaluated to 1.0 when sitting still or lying still, and the gain of the Kalman filter selects a gain 1.0 in the value scope [0, 1.0]; when in motion, if the heart rate signal quality indicator satisfies a requirement, the heart rate effective indicator corresponding to the first action amount is evaluated to 0.8, and the gain of the Kalman filter is determined as 0.8. An embodiment that the heart rate signal quality indicator is ineligible in motion will be expounded infra.

S3087: performing Kalman filtering to the transition value of the heart rate based on the gain of the Kalman filter within the first estimation scope, and using a filtered output as the estimation value of the heart rate.

In another embodiment, when the heart rate signal quality indicator is ineligible:

S30813: when the heart rate signal quality indicator is ineligible, obtaining a heart rate prediction value corresponding to the first action amount based on a linear fitting or a non-linear fitting between the action amount and the heart rate;

The fitting approach may refer to previous Examples 1, 2 and the above description about fitting.

S30814: updating the reference heart rate value and using the heart rate prediction value as the updated reference heart rate value;

S30815: reducing the heart rate effective indicator corresponding to the first action amount and using it as the gain of the Kalman filtering;

Exemplarily, as mentioned above, in motion, when the heart rate signal quality indicator is eligible, the heart rate effective indicator corresponding to the first action amount is evaluated to 0.8, and the gain of the Kalman filtering is determined to be 0.8; if the signal quality indicator is ineligible, the heart rate effective indicator in the step S30815 may be reduced to 0.7, and the gain of the Kalman filter is determined to 0.7.

S30816: within the first estimation scope, performing Kalman filtering to the updated reference heart rate value based on the gain of the Kalman filtering, and using a filtered output as the estimation value of the heart rate.

In another embodiment,

When an absolute value of a difference between the heart rate prediction value and an action reference heart rate corresponding to the first action amount is greater than a ninth threshold, the heart rate prediction value is calibrated. Exemplarily, there are a lot of approaches to calibrate: the action reference heart rate may be directly used as the heart rate prediction value; the absolute value may be directly calibrated to be less than the ninth threshold; or the preset calibration empirical value may be directly used as the heart rate prediction value; or any appropriate prior calibration method may be adopted; or even the linear fitting or non-linear fitting between the action amount and the heart rate may be calibrated.

In a dynamic state (i.e., when a relatively apparent action amount is existent), by searching a motion jump point search, the reference heart rate value corresponding to the first action amount (or the motion frequency corresponding to the first action amount) may be obtained, which is also referred to as an action reference heart rate value.

This embodiment will be illustrated in detail below:

A motion jump point refers to a signal position point of the reference heart rate value obtained when the heart rate signal quality is relatively high. In the frequency domain, the frequency value at the motion jump point may be used as the reference heart rate value.

The motion jump point may be determined through the following solution:

determining a motion frequency peak of a PPG (photoplethysmography) signal frequency domain based on the motion frequency peak of the motion signal frequency domain, determining a non-motion frequency peak (which will be explained in detail infra), and further comparing the PPG frequency domain features before and after adaptive filtering; if the frequency of the largest non-motion frequency peak is close, it may be determined as the heart rate frequency peak; if the signal quality of the heart rate frequency peak is eligible, the position of the segment of PPG signal is determined as the motion jump point.

In detail, the action reference heart rate value is obtained through the following step in another embodiment:

S5011: sampling at the same time point to obtain a PPG signal and a motion signal of the same time length;

S5012: obtaining a first spectrogram based on the motion signal, and obtaining frequency point values corresponding to the largest peak and second largest peak in the first spectrogram;

S5013: obtaining a second spectrogram based on the PPG signal and meanwhile performing adaptive filtering to the PPG signal to obtain a third spectrogram;

S5014: determining the largest peak, the second largest peak, and the third largest peak in the second spectrogram, obtaining frequency point values corresponding to the largest peak, the second largest peak, and the third largest peak, and obtaining an amplitude of the third largest peak;

S5015: in the frequency point values obtained in step S5014, if a frequency point value whose differences from each frequency point value obtained in step S5012 are beyond a set error scope, determining the frequency peak corresponding to the frequency point value as a non-motion frequency peak;

S5016: determining frequency peaks greater than or equal to the amplitude in the third spectrogram based on the amplitude determined in step S5014 to obtain frequency points corresponding the frequency peaks; and if a frequency point value whose difference from the each frequency point value obtained in step S5012 is beyond the set error scope, determining that the frequency peak corresponding to the frequency point value as a non-motion frequency peak;

S5017: based on the non-motion frequency peaks determined in the second spectrogram and the third spectrogram, if the frequency difference between the largest non-motion frequency peaks in the second frequency spectrogram and the third frequency spectrogram is within a set error scope, obtaining the amplitude of the non-motion frequency peak in the second spectrogram;

S5018: computing a ratio of the amplitude obtained in step S5017 to a sum of amplitudes of all sampling frequency points in the second spectrogram;

S5019: if the ratio is greater than or equal to a tenth threshold, determining that the position of the currently obtained sampling signal segment is the motion jump point;

S5020: after obtaining the motion jump point, determining its corresponding frequency point value using the amplitude of the point, and determining its corresponding frequency value based on the frequency point as the action reference heart rate value.

Hereinafter, the motion jump point will be further illustrated in detail with reference to FIGS. 6-8.

Particularly, the horizontal axis in FIGS. 6-8 represents frequency point values, and the longitudinal axis represents amplitudes.

1) obtaining an original PPG (photoplethysmography) signal and a motion signal (time domain) of the same length after starting sampling at time t1;

2) performing Fourier transformation to the original motion signal to obtain a first spectrogram, which is a frequency domain diagram of the motion signal, as shown in FIG. 6. The largest peak and second largest peak in the first spectrogram represent motion frequency peaks, and the frequency point values of the two motion frequency peaks are determined.

Particularly, the frequency point values are values at the horizontal axis in the figure; an interval between two neighboring frequency point values corresponds to an interval or resolution of the frequency axis, and the frequency point values correspond to frequency values. The frequency interval or resolution of the frequency axis is dependent on the frequency of sampling the motion signal and the sampling point number when performing Fourier transformation to the time domain, wherein the frequency point values are denoted as fb, the sampling frequency is denoted as fs, and the sampling point number is denoted as Nr.

The translation relationship between a frequency f and the frequency point value: f=fb×(fs/Nr),

translating into a heart rate value according to a certain frequency value: HR=f×60,

if the sampling frequency fs of the PPG signal is 25 Hz, the Nr upon Fourier transformation is 512, and the frequency point value fb of the heart rate frequency peak is 38, the heart rate is 38×(25/512)×60≈111.

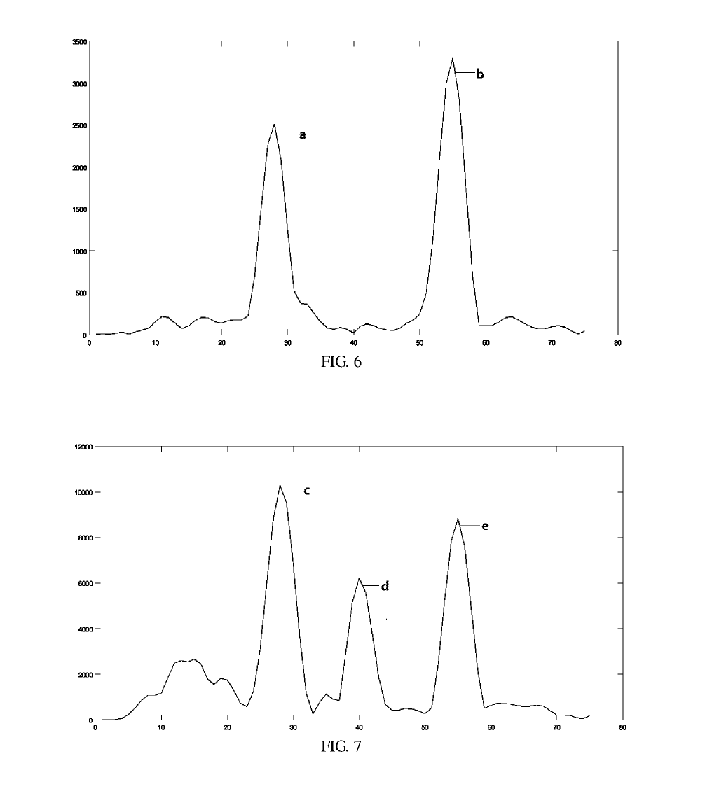

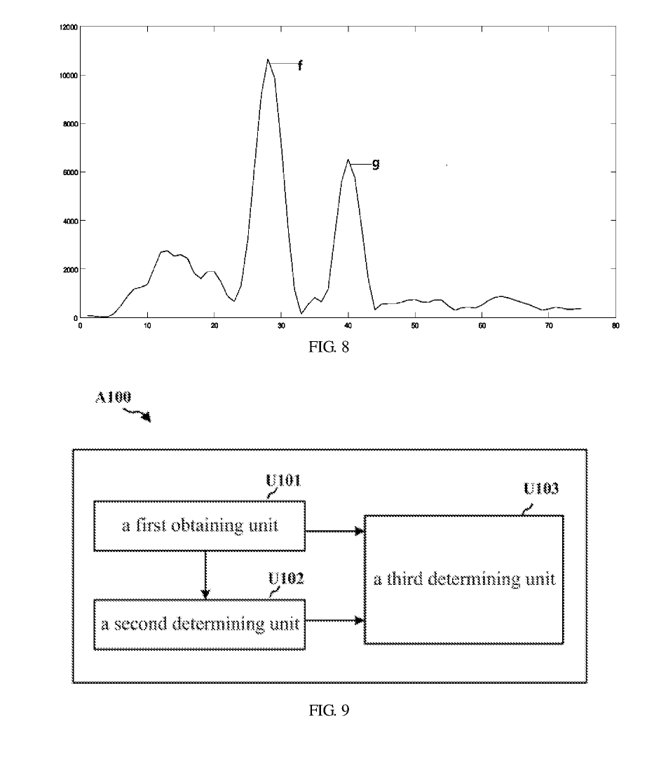

3) performing Fourier transformation to the original PPG signal to obtain a second spectrogram, i.e., the frequency domain spectrogram of the original PPG signal, as shown in FIG. 7. Meanwhile, the original PPG signal is subjected to adaptive filtering, and then the adaptively filtered signal is subjected to Fourier transformation to obtain a third spectrogram, i.e., the frequency domain spectrogram of the filtered PPG signal, as shown in FIG. 8.

n relatively large peaks in the second spectrogram are selected, preferably, n=3, the three peaks being the largest peak, the second largest peak, and the third largest peak, respectively; as shown in FIG. 7, peaks c, d, e are 3 relatively large peaks in the spectrum, and the amplitude of the third largest peak is determined, denoted as Amplitude.

4) comparing features of the first spectrogram and the second spectrogram. The frequency point values of peaks c and e are closer to the frequency point values of peaks a and b in the first spectrum, respectively, i.e., within the set error scope; the frequency point value of peak d is different from that of peak a and peak b, i.e., beyond the set error scope; then peak c and peak e are determined as motion frequency peaks, and peak d is determined as a non-motion frequency peak, which is possibly the heart rate frequency peak.

5) comparing the feature of relatively large peaks whose amplitudes≥Amplitude in the third spectrogram with the feature of the motion spectrum peak of the first spectrogram; the frequency point value of peak f is close to that of peak a, i.e., within a set error range; the frequency point value of peak g is different from that of peak a and peak b, i.e., beyond the set error scope; then, it is determined that the peak f is the motion frequency peak, and peak g is the non-motion frequency peak.

6) comparing peak c, peak d, and peak e of the second spectrogram and the relatively large peaks (namely, peak f, peak g) whose amplitudes≥Amplitude in the third spectrogram, wherein the frequency point values of peaks c and f are close, i.e., within the set error scope, such that they are the same motion frequency peak; the frequency point value of peak d is close to that of peak f, i.e., within the set error scope; then, peak d and peak g are determined as the heart rate frequency peak.

7) calculating the ratio z of the amplitude of peak d to the sum of the amplitudes of all frequency point values in the second spectrogram; if the ratio z≥the tenth threshold, it may be believed that the position of the sampling signal segment at time t1 is the motion jump point. The tenth threshold is an empirical value, preferably 0.03.

The frequency point value corresponding to the amplitude of peak d is determined; the frequency value is translated to the heart rate value; and the reference heart rate value is further determined based on the heart rate value; the current reference heart rate value is the action reference heart rate value.

After determining the action reference heart rate value in the dynamic state, the action amount indicated by the motion signal sampled at time t1 is obtained; if the action amount is 300 and the searched heart rate value is 95 times per minute, it may be deemed that when the action amount is 300, the action reference heart rate value of its corresponding user is 95 times per minute. At this point, the correspondence relationship between a certain action amount and an action reference heart rate value has been determined.

In a motion scenario, after the action reference heart rate value is found, the heart rate prediction value may be calculated based on a fitting equation between the action amount and the heart rate; the heart rate prediction value is compared with the action reference heart rate value; if the absolute value of the difference between the heart rate prediction value and the action reference heart rate value is greater than or equal to the ninth threshold, the heart rate prediction value is calibrated; otherwise, no calibration is made.

It needs to be noted that various thresholds or threshold intervals disclosed by the present disclosure may be obtained based on empirical values, experiment statistics, or fitting, and the like.

In addition, besides the method of estimating a cardiovascular characteristic parameter described above, the present disclosure further discloses a corresponding apparatus for estimating a cardiovascular characteristic parameter.

Referring to FIG. 9, in one embodiment, the apparatus A100 comprises:

a first obtaining unit U101, configured to obtain a first action amount of a subject and a sensing signal of a cardiovascular aspect within a first time interval, wherein the first action amount is located within a first action amount threshold range, and the sensing signal of the cardiovascular aspect includes any one or a combination of: a photoplethysmographic signal, an electrocardiographic signal;

a second determining unit U102, configured to determine, based on the first action amount threshold range, a first estimation scope for estimating the cardiovascular characteristic parameter; and

a third determining unit U103, configured to determine an estimation value of the cardiovascular characteristic parameter based on the above unit.

For example, the first obtaining unit can be PPG sensor and/or ECG sensor. The second determining unit and the third determining unit can be different processors or same processor.

In another embodiment, the cardiovascular characteristic parameter includes, but not limited to: a heart rate, a respiratory rate, an oxyhemoglobin saturation, a heart rate variability (HRV), and a blood pressure instant variability (BPIV).

In another unit, the first action amount includes any one of the following parameters or a combination thereof: an acceleration parameter, a velocity parameter, pace count, and a pace frequency parameter.

In another embodiment, the third determining unit specifically comprises:

a first subunit configured to: when a reliability degree of the sensing signal of the cardiovascular aspect does not meet a first threshold requirement, determine a cardiovascular characteristic reference value corresponding to the first action amount based on the first estimation scope; and

a second subunit configured to: use the cardiovascular characteristic reference value corresponding to the first action amount as an estimation value of the cardiovascular characteristic parameter.

In a further embodiment, the third determining unit specifically comprises:

a third subunit configured to: when the reliability degree of the sensing signal of the cardiovascular aspect satisfies the first threshold requirement, determine, based on the first estimation scope, a cardiovascular characteristic parameter baseline value corresponding to the first action amount threshold range and a cardiovascular character parameter variable corresponding to the first action amount, wherein the cardiovascular characteristic parameter baseline value is defined different from the cardiovascular character parameter variable; and

a fourth subunit configured to: determine the estimation value of the cardiovascular characteristic parameter based on the cardiovascular character parameter variable and the cardiovascular characteristic parameter baseline value.

In a further embodiment, the third determining unit specifically comprises:

a fifth subunit configured to: when the reliability degree of the sensing signal of the cardiovascular aspect satisfies a second threshold requirement, determine a computation equation corresponding to the first estimation scope and respective parameters of the equation, wherein at least one parameter of the equation corresponds to the first action amount; and

a sixth subunit configured to: determine the estimation value of the cardiovascular characteristic parameter based on the computation equation.

In a further embodiment, the third determining unit specifically comprises:

a seventh subunit configured to determine a cardiovascular characteristic reference value corresponding to the first action amount based on the first estimation scope when a change ratio of the first action amount within a second time interval to the preceding action amount is within a third threshold range; and

an eighth subunit configured to use the cardiovascular characteristic reference value corresponding to the first action amount as the estimation value of the cardiovascular characteristic parameter.

In a further embodiment, the third determining unit specifically comprises:

a ninth subunit configured to: when a change ratio of the first action amount within a second time interval to the preceding action amount is within a fourth threshold range, determine a computation equation corresponding to the first estimation scope and respective parameters of the equation, wherein at least one parameter of the equation corresponds to the first action amount; and

a tenth subunit configured to determine the estimation value of the cardiovascular characteristic parameter based on the computation equation.

In a further embodiment, the third determining unit specifically comprises:

an eleventh subunit configured to: when an absolute value of a difference between a largest value and a minimum value of the sensing signal of the cardiovascular aspect within the second time interval is within a fifth threshold range, determine a cardiovascular characteristic reference value corresponding to the first action amount based on the first estimation scope; and

a twelfth subunit configured to use the cardiovascular characteristic reference value corresponding to the first action amount as an estimation value of the cardiovascular characteristic parameter.

In a further embodiment, the third determining unit specifically comprises:

a thirteenth subunit configured to: when an absolute value of a difference between a largest value and a minimum value of the sensing signal of the cardiovascular aspect within the second time interval is within a sixth threshold range, determine a computation equation corresponding to the first estimation scope and respective parameters of the equation, wherein at least one parameter of the equation corresponds to the first action amount; and

a fourteenth subunit configured to determine the estimation value of the cardiovascular characteristic parameter based on the calculation equation.

In another embodiment: the upper limit and/or lower limit of the first estimation scope have an attribute of varying with the first action amount.

In another embodiment, data in the first estimation scope has an attribute of updating with the estimation value of the cardiovascular characteristic parameter.

In another embodiment, the apparatus further comprises:

a fourth classifying unit configured to classify the estimation value of the cardiovascular characteristic parameter.

In another embodiment, the apparatus further comprises:

a fifth determining unit configured to: when a change ratio of multiple historical data of the estimation value of the cardiovascular characteristic parameter is within a seventh threshold range, determine the estimation value of the cardiovascular characteristic parameter every the third time interval at least based on the preceding historical data of the estimation value of the cardiovascular characteristic parameter; wherein the sensing signal of the cardiovascular aspect is not obtained at the third time interval period.

In a further embodiment, the apparatus further comprises:

a sixth estimating unit configured to estimate a time interval of obtaining the sensing signal of the cardiovascular aspect at the next time based on the estimation value of the cardiovascular characteristic parameter.

In a further embodiment, the apparatus further comprises:

a seventh estimating unit configured to estimate the next time of switching on or switching off the following sensor based on the estimation value of the cardiovascular characteristic parameter: the sensor for obtaining the sensing signal of the cardiovascular aspect.

In a further embodiment, the cardiovascular characteristic parameter baseline value corresponding to the first action amount threshold range has an attribute of being updated with the estimation value of the cardiovascular characteristic parameter.

In a further embodiment, the apparatus further comprises:

an eighth calibration unit configured to: when a difference (or an absolute value of the difference) between the estimation of the cardiovascular characteristic parameter and the cardiovascular characteristic parameter baseline value corresponding to the first action amount threshold range is greater than an eighth threshold, calibrate the cardiovascular characteristic parameter baseline value corresponding to the first action amount threshold range using the estimation value of the cardiovascular characteristic parameter, causing the absolute value of the difference between the estimation of the cardiovascular characteristic parameter and the cardiovascular characteristic parameter baseline value corresponding to the first action amount threshold range not greater than the eighth threshold.

In a further embodiment, the third determining unit specifically comprises:

a fifteenth subunit configured to: when the sensing signal of the cardiovascular aspect is a PPG signal and the cardiovascular characteristic parameter is a heart rate, compute the heart rate directly using the PPG signal and using the calculated heart rate as a reference heart rate value;

a sixteenth subunit configured to: divide an amplitude of a frequency point value in a spectrogram corresponding to the reference heart rate by the sum of all frequency point amplitudes, a resulting ratio being used as the heart rate signal quality indicator;

a seventeenth subunit configured to: when the heart rate signal quality indicator satisfies a requirement, determine a motion frequency based on the first action amount;

an eighteenth subunit configured to perform adaptive filtering to the PPG signal using the motion frequency;

a nineteenth subunit configured to compute the heart rate based on the filtered signal, used as a transition value of the heart rate;

a twentieth subunit configured to determine a gain of Kalman filtering based on a heart rate effective indicator corresponding to the first action amount;

a twenty-first subunit configured to perform Kalman filtering to the transition value of the heart rate based on the gain of the Kalman filter within the first estimation scope, and use a filtered output as the estimation value of the heart rate.

In a further embodiment, the third determining unit specifically comprises:

a twenty-second subunit configured to: when the sensing signal of the cardiovascular aspect is a PPG signal and the cardiovascular characteristic parameter is a heart rate, compute the heart rate directly using the PPG signal and using the calculated heart rate as a reference heart rate value;

a twenty-third subunit configured to: divide an amplitude of a frequency point value in a spectrogram corresponding to the reference heart rate by the sum of all frequency point amplitudes, a resulting ratio being used as the heart rate signal quality indicator;

a twenty-fourth subunit configured to: when the heart rate signal quality indicator is ineligible, obtain a heart rate prediction value corresponding to the first action amount based on a linear fitting or a non-linear fitting between the action amount and the heart rate;

a twenty-fifth subunit configured to: update the reference heart rate value and using the heart rate prediction value as the updated reference heart rate value;

a twenty-sixth subunit configured to reduce the heart rate effective indicator corresponding to the first action amount and using it as the gain of the Kalman filtering; and

a twenty-seventh subunit configured to: within the first estimation scope, perform Kalman filtering to the updated reference heart rate value based on the gain of the Kalman filtering, and use a filtered output as the estimation value of the heart rate.

In another embodiment, the apparatus further comprises a twenty-eighth subunit, configured to:

when an absolute value of a difference between the heart rate prediction value and an action reference heart rate corresponding to the first action amount is greater than a ninth threshold, calibrate the heart rate prediction value.

In a further embodiment, the apparatus further comprises a twenty-ninth subunit configured to obtain an action reference heart rate value, and the twenty-ninth subunit comprising:

a first module configured to sample at the same time point to obtain a PPG signal and a motion signal of the same time length;

a second module configured to obtain a first spectrogram based on the motion signal, and obtaining frequency point values corresponding to the largest peak and second largest peak in the first spectrogram;

a third module configured to obtain a second spectrogram based on the PPG signal and meanwhile performing adaptive filtering to the PPG signal to obtain a third spectrogram;

a fourth module configured to determine the largest peak, the second largest peak, and the third largest peak in the second spectrogram, obtain frequency point values corresponding to the largest peak, the second largest peak, and the third largest peak, and obtain an amplitude of the third largest peak;

a fifth module configured to: in the frequency point values obtained in the fourth module, if a frequency point value whose differences from each frequency point value obtained in the second module are beyond a set error scope, determine the frequency peak corresponding to the frequency point value as a non-motion frequency peak;

a sixth module configured to determine, based on the amplitude determined in the fourth module, frequency peaks greater than or equal to the amplitude in the third spectrogram, obtain frequency points corresponding the frequency peaks; and if a frequency point value whose difference from the each frequency point value obtained in the second module is beyond the set error scope, determine that the frequency peak corresponding to the frequency point value as a non-motion frequency peak;

a seventh module configured to: based on the non-motion frequency peaks determined in the second spectrogram and the third spectrogram, if the frequency difference between the largest non-motion frequency peaks in the second frequency spectrogram and the third frequency spectrogram is within a set error scope, obtain the amplitude of the non-motion frequency peak in the second spectrogram;

an eighth module configured to compute a ratio of the amplitude obtained in seventh module to a sum of amplitudes of all sampling frequency points in the second spectrogram;

a ninth module configured to: if the ratio is greater than or equal to a tenth threshold, determine that the position of the currently obtained sampling signal segment is the motion jump point; and

a tenth module configured to: after obtaining the motion jump point, determine its corresponding frequency point value using the amplitude of the point, and determine its corresponding frequency value based on the frequency point as the action reference heart rate value.

In another embodiment, the apparatus is a processor, or a sensor, or a wearable device (for example, smart watch or smart wristband), or a terminal (for example, smart phone or medical equipment, or physiological or exercise monitoring equipment). When the apparatus is a processor, the processor can implement each unit of the units of the foregoing embodiments through a plurality of logic units or one logic unit in the processor; when the apparatus is a sensor, the sensor may include a processor and a sensing unit, wherein the sensing unit is used to sense and collect the signal, and the processor is used to implement each unit of the above embodiments through the logic unit therein; and when the apparatus is a terminal, the terminal includes the sensor and/or processor. Understandably, the processor here may also be implemented by instructing (set) to execute the method in the preceding embodiment.

In addition, the present disclosure further discloses a computer-readable storage medium, wherein:

the computer-readable storage medium comprises one or more programs, the one or more programs being for executing any method above.

Further, the present disclosure also discloses a data processing device, the data processing device comprising:

the computer-readable storage medium mentioned above; and

one or more processors for executing programs in the computer-readable storage medium.

The foregoing description of the exemplary embodiments of the present disclosure has been presented only for the purposes of illustration and description and is not intended to be exhaustive or to limit the disclosure to the precise forms disclosed. Many modifications and variations are possible in light of the above teaching.

The embodiments above only schematically illustrate the principle of the present disclosure. It should be understood that the modifications and alterations of the arrangements and the details described herein will be obvious to those skilled in the art. Therefore, the present disclosure is not intended to be limited by the scope of the claims, not limited to the specific details of the present disclosure provided to illustrate and describe the embodiments.

The embodiments were chosen and described in order to explain the principles of the disclosure and their practical application so as to activate others skilled in the art to utilize the disclosure and various embodiments and with various modifications as are suited to the particular use contemplated. Alternative embodiments will become apparent to those skilled in the art to which the present disclosure pertains without departing from its spirit and scope. Accordingly, the scope of the present disclosure is defined by the appended claims rather than the foregoing description and the exemplary embodiments described therein.

What is claimed is:

1. An apparatus for estimating a cardiovascular characteristic parameter comprising:
a first obtaining unit configured to obtain a first action amount of a subject and a sensing signal of a cardiovascular aspect within a first time interval, wherein the first action amount is located within a first action amount threshold range, and the sensing signal of the cardiovascular aspect includes any one or a combination of: a photoplethysmographic signal and an electrocardiographic signal;
a second determining unit configured to determine, based on the first action amount threshold range, a first estimation scope for estimating the cardiovascular characteristic parameter; and
a third determining unit configured to determine an estimation value of the cardiovascular characteristic parameter based on the first obtaining unit and the second determining unit.

2. The apparatus according to claim 1, wherein the cardiovascular characteristic parameter comprises: a heart rate, a respiratory rate, an oxyhemoglobin saturation, a heart rate variability (HRV), and a blood pressure instant variability (BPIV).

3. The apparatus according to claim 1, wherein the first action amount comprises at least one selected from the group consisting of an acceleration parameter, a velocity parameter, pace count, and a pace frequency parameter.

4. The apparatus according to claim 1, wherein the third determining unit comprises:
a first subunit configured to: when a reliability degree of the sensing signal of the cardiovascular aspect does not meet a first threshold requirement, determine a cardiovascular characteristic reference value corresponding to the first action amount based on the first estimation scope; and
a second subunit configured to: use the cardiovascular characteristic reference value corresponding to the first action amount as an estimation value of the cardiovascular characteristic parameter.

5. The apparatus according to claim 1, wherein the third determining unit comprises:
a third subunit configured to: when the reliability degree of the sensing signal of the cardiovascular aspect satisfies the first threshold requirement, determine, based on the first estimation scope, a cardiovascular characteristic parameter baseline value corresponding to the first action amount threshold range and a cardiovascular character parameter variable corresponding to the first action amount, wherein the cardiovascular characteristic parameter baseline value is defined different from the cardiovascular character parameter variable; and
a fourth subunit configured to: determine the estimation value of the cardiovascular characteristic parameter based on the cardiovascular character parameter variable and the cardiovascular characteristic parameter baseline value.

6. The apparatus according to claim 1 wherein the third determining unit comprises:
a fifth subunit configured to: when the reliability degree of the sensing signal of the cardiovascular aspect satisfies a second threshold requirement, determine a computation equation corresponding to the first estimation scope and respective parameters of the equation, wherein at least one parameter of the equation corresponds to the first action amount; and
a sixth subunit configured to: determine the estimation value of the cardiovascular characteristic parameter based on the computation equation.

7. The apparatus according to claim 1, wherein the third determining unit comprises:
a seventh subunit configured to determine a cardiovascular characteristic reference value corresponding to the first action amount based on the first estimation scope when a change ratio of the first action amount within a second time interval to the preceding action amount is within a third threshold range; and
an eighth subunit configured to use the cardiovascular characteristic reference value corresponding to the first action amount as the estimation value of the cardiovascular characteristic parameter.

8. The apparatus according to claim 1, wherein the third determining unit comprises:
a ninth subunit configured to: when a change ratio of the first action amount within a second time interval to the preceding action amount is within a fourth threshold range, determine a computation equation corresponding to the first estimation scope and respective parameters of the equation, wherein at least one parameter of the equation corresponds to the first action amount; and
a tenth subunit configured to determine the estimation value of the cardiovascular characteristic parameter based on the computation equation.

9. The apparatus according to claim 1, wherein the third determining unit comprises:
an eleventh subunit configured to: when an absolute value of a difference between a largest value and a minimum value of the sensing signal of the cardiovascular aspect within the second time interval is within a fifth threshold range, determine a cardiovascular characteristic reference value corresponding to the first action amount based on the first estimation scope; and
a twelfth subunit configured to use the cardiovascular characteristic reference value corresponding to the first action amount as an estimation value of the cardiovascular characteristic parameter.

10. The apparatus according to claim 1, wherein the third determining unit comprises:
a thirteenth subunit configured to: when an absolute value of a difference between a largest value and a minimum value of the sensing signal of the cardiovascular aspect within the second time interval is within a sixth threshold range, determine a computation equation corresponding to the first estimation scope and respective parameters of the equation, wherein at least one parameter of the equation corresponds to the first action amount; and
a fourteenth subunit configured to determine the estimation value of the cardiovascular characteristic parameter based on the calculation equation.

11. The apparatus according to claim 1, further comprising:
a fourth classifying unit configured to classify the estimation value of the cardiovascular characteristic parameter.

12. The apparatus according to claim 1, further comprising:
a fifth determining unit configured to: when a change ratio of multiple historical data of the estimation value of the cardiovascular characteristic parameter is within a seventh threshold range, determine the estimation value of the cardiovascular characteristic parameter every the third time interval at least based on the preceding historical data of the estimation value of the cardiovascular characteristic parameter;
wherein the sensing signal of the cardiovascular aspect is not obtained at the third time interval period.

13. The apparatus according to claim 1, further comprising:
a sixth estimating unit configured to estimate a time interval of obtaining the sensing signal of the cardiovascular aspect at the next time based on the estimation value of the cardiovascular characteristic parameter.

14. The apparatus according to claim 1, further comprising:
a seventh estimating unit configured to estimate the next time of switching on or switching off the following sensor based on the estimation value of the cardiovascular characteristic parameter: the sensor for obtaining the sensing signal of the cardiovascular aspect.

15. The apparatus according to claim 1, further comprising:
an eighth calibration unit configured to: when a difference (or an absolute value of the difference) between the estimation of the cardiovascular characteristic parameter and the cardiovascular characteristic parameter baseline value corresponding to the first action amount threshold range is greater than an eighth threshold, calibrate the cardiovascular characteristic parameter baseline value corresponding to the first action amount threshold range using the estimation value of the cardiovascular characteristic parameter, causing the absolute value of the difference between the estimation of the cardiovascular characteristic parameter and the cardiovascular characteristic parameter baseline value corresponding to the first action amount threshold range not greater than the eighth threshold.

16. The apparatus according to claim 1, wherein the third determining unit comprises:
a fifteenth subunit configured to: when the sensing signal of the cardiovascular aspect is a PPG signal and the cardiovascular characteristic parameter is a heart rate, compute the heart rate directly using the PPG signal and using the calculated heart rate as a reference heart rate value;
a sixteenth subunit configured to: divide an amplitude of a frequency point value in a spectrogram corresponding to the reference heart rate by the sum of all frequency point amplitudes, a resulting ratio being used as the heart rate signal quality indicator;
a seventeenth unit configured to: when the heart rate signal quality indicator satisfies a requirement, determine a motion frequency based on the first action amount;
an eighteenth unit configured to perform adaptive filtering to the PPG signal using the motion frequency;
a nineteenth unit configured to compute the heart rate based on the filtered signal, used as a transition value of the heart rate;
a twentieth unit configured to determine a gain of Kalman filtering based on a heart rate effective indicator corresponding to the first action amount;
a twenty-first subunit configured to perform Kalman filtering to the transition value of the heart rate based on the gain of the Kalman filter within the first estimation scope, and use a filtered output as the estimation value of the heart rate.

17. The apparatus according to claim 1, wherein the third determining unit comprises:
a twenty-second subunit configured to: when the sensing signal of the cardiovascular aspect is a PPG signal and the cardiovascular characteristic parameter is a heart rate, compute the heart rate directly using the PPG signal and using the calculated heart rate as a reference heart rate value;
a twenty-third subunit configured to: divide an amplitude of a frequency point value in a spectrogram corresponding to the reference heart rate by the sum of all frequency point amplitudes, a resulting ratio being used as the heart rate signal quality indicator;
a twenty-fourth subunit configured to: when the heart rate signal quality indicator is ineligible, obtain a heart rate prediction value corresponding to the first action amount based on a linear fitting or a non-linear fitting between the action amount and the heart rate;
a twenty-fifth subunit configured to: update the reference heart rate value and using the heart rate prediction value as the updated reference heart rate value;
a twenty-sixth subunit configured to reduce the heart rate effective indicator corresponding to the first action amount and using it as the gain of the Kalman filtering; and
a twenty-seventh subunit configured to: within the first estimation scope, perform Kalman filtering to the updated reference heart rate value based on the gain of the Kalman filtering, and use a filtered output as the estimation value of the heart rate.

18. The apparatus according to claim 17, further comprising:
a twenty-eighth subunit, configured to:
when an absolute value of a difference between the heart rate prediction value and an action reference heart rate corresponding to the first action amount is greater than a ninth threshold, calibrate the heart rate prediction value.

19. The apparatus according to claim 18, further comprising:
a twenty-ninth subunit configured to obtain an action reference heart rate value, wherein the twenty-ninth subunit comprises:
a first module configured to sample at the same time point to obtain a PPG signal and a motion signal of the same time length;
a second module configured to obtain a first spectrogram based on the motion signal, and obtaining frequency point values corresponding to the largest peak and second largest peak in the first spectrogram;
a third module configured to obtain a second spectrogram based on the PPG signal and meanwhile performing adaptive filtering to the PPG signal to obtain a third spectrogram;
a fourth module configured to determine the largest peak, the second largest peak, and the third largest peak in the second spectrogram, obtain frequency point values corresponding to the largest peak, the second largest peak, and the third largest peak, and obtain an amplitude of the third largest peak;
a fifth module configured to: in the frequency point values obtained in the fourth module, if a frequency point value whose differences from each frequency point value obtained in the second module are beyond a set error scope, determine the frequency peak corresponding to the frequency point value as a non-motion frequency peak;
a sixth module configured to determine, based on the amplitude determined in the fourth module, frequency peaks greater than or equal to the amplitude in the third spectrogram, obtain frequency points corresponding the frequency peaks; and if a frequency point value whose difference from the each frequency point value obtained in the second module is beyond the set error scope, determine that the frequency peak corresponding to the frequency point value as a non-motion frequency peak;
a seventh module configured to: based on the non-motion frequency peaks determined in the second spectrogram and the third spectrogram, if the frequency difference between the largest non-motion frequency peaks in the second frequency spectrogram and the third frequency spectrogram is within a set error scope, obtain the amplitude of the non-motion frequency peak in the second spectrogram;
an eighth module configured to compute a ratio of the amplitude obtained in seventh module to a sum of amplitudes of all sampling frequency points in the second spectrogram;
a ninth module configured to: if the ratio is greater than or equal to a tenth threshold, determine that the position of the currently obtained sampling signal segment is the motion jump point; and
a tenth module configured to: after obtaining the motion jump point, determine its corresponding frequency point value using the amplitude of the point, and determine its corresponding frequency value based on the frequency point as the action reference heart rate value.

20. The apparatus according to claim 1, wherein the apparatus is a processor, or a sensor, or a wearable device, or a terminal.
